SEQUENCE LISTING (22 sequences)

SEQ ID NO: 1 - DNA, 846 bp, Arabidopsis thaliana
  CDS (1)..(843)
atggataccgagtttctccgaaccctagatcgtcagattcttttgggtgtcttcgttgctttcgtcgccgttggtgctggtgctgcttattttcttacatcctccaagaaacgcagagtgtgtttggatccagagaatttcaaggagttcaagcttgttaagagacatcagcttagtcacaatgtggccaagttcgtttttgaactcccaacttctacttctgtgttgggtcttcccattggacaacacatcagttgcaggggaaaggatggtcaaggagaggatgttattaagccatacaccccgactacgttagactctgacgttggacgtttcgaacttgtcattaagatgtatccgcaaggacggatgtctcatcatttcagggagatgcgtgttggagaccatcttgccgtaaagggaccaaagggtaggttcaagtatcaaccaggtcagtttagggcatttggaatgcttgctggaggttcaggcatcactcccatgttccaagtggccagagcaattctagaaaacccaacagacaagacaaaggtgcatctcatttacgccaacgtcacatacgacgacattctcttgaaggaagaattggagggtcttactaccaattaccctgaacaatttaaaatcttctatgttttgaaccagcctccggaagtatgggatggtggtgttggatttgtatcaaaggaaatgattcagactcattgccctgcacctgcatctgatattcagatcctaagatgcggaccaccgccaatgaacaaggccatggctgcaaaccttgaagctctgggatactctccggagatgcaattccagttctga

SEQ ID NO: 2 - protein, 281 aa, Arabidopsis thaliana
MDTEFLRTLDRQILLGVFVAFVAVGAGAAYFLTSSKKRRVCLDPENFKEFKLVKRHQLSHNVAKFVFELPTSTSVLGLPIGQHISCRGKDGQGEDVIKPYTPTTLDSDVGRFELVIKMYPQGRMSHHFREMRVGDHLAVKGPKGRFKYQPGQFRAFGMLAGGSGITPMFQVARAILENPTDKTKVHLIYANVTYDDILLKEELEGLTTNYPEQFKIFYVLNQPPEVWDGGVGFVSKEMIQTHCPAPASDIQILRCGPPPMNKAMAANLEALGYSPEMQFQF

SEQ ID NO: 3 - DNA, 729 bp, Nicotiana tabacum
  CDS (1)..(726)
gtatgcttggatcctgagaggttcaaggaatttaagcttgtgaagcgtacacaaataagccacaatgttgcaaagttcagatttgaactccccacacctacttctgtattgggcctacccattggacaacatattagttgcaggggcaaggatagtcaaggtgaagaggttgttaaaccgtacacaccaactactttggattcagatgttggatattttgaactagttattaagatgtatcctcaaggaaggatgtctcatcatttccgagaaatgcgtgagggtgattatttggctgtgaagggacctaagggccgctttaagtaccagcctggccaagtgagagcatttggaatgcttgctggaggctctggcattaccccaatgtttcaggttgctagagctattctcgaaaatccaaatgacaagacaaaggtgcacttgatatatgctaatgttacctatgaagacatacttttaaaggaacagttggatggccttgctgctaactatcctgaccgtttcaaaatttattacgtactgaatcagcctcctgaagtatggagcggtggtgttggatttgtgtccaaggaaatgattcagactcattgtcctgccccggcatctgacattcagatactgaggtgtggtccacctccaatgaacaaggctatggctgctcatcttgaagcccttggatacaccccagagatgcaattccagttttaa

SEQ ID NO: 4 - protein, 242 aa, Nicotiana tabacum
VCLDPERFKEFKLVKRTQISHNVAKFRFELPTPTSVLGLPIGQHISCRGKDSQGEEVVKPYTPTTLDSDVGYFELVIKMYPQGRMSHHFREMREGDYLAVKGPKGRFKYQPGQVRAFGMLAGGSGITPMFQVARAILENPNDKTKVHLIYANVTYEDILLKEQLDGLAANYPDRFKIYYVLNQPPEVWSGGVGFVSKEMIQTHCPAPASDIQILRCGPPPMNKAMAAHLEALGYTPEMQFQF

SEQ ID NO: 5 - DNA, 32 bp, Artificial Sequence
  Description of Artificial Sequence Synthetic Primer
tatacccgggatggataccgagtttctccgaa

SEQ ID NO: 6 - DNA, 32 bp, Artificial Sequence
  Description of Artificial Sequence Synthetic Primer
tatacccggggaactggaattgcatctccgga

SEQ ID NO: 7 - DNA, 20 bp, Artificial Sequence
  Description of Artificial Sequence Synthetic Primer
gctatgaccatgattacgcc

SEQ ID NO: 8 - DNA, 18 bp, Artificial Sequence
  Description of Artificial Sequence Synthetic Primer
tgagacatccgtccttgc

SEQ ID NO: 9 - DNA, 20 bp, Artificial Sequence
  Description of Artificial Sequence Synthetic Primer
acgttgtaaaacgacggcca

SEQ ID NO: 10 - DNA, 19 bp, Artificial Sequence
  Description of Artificial Sequence Synthetic Primer
ccgactacgttagactctg

SEQ ID NO: 11 - DNA, 21 bp, Artificial Sequence
  Description of Artificial Sequence Synthetic Primer
ttcgttgctttcgtcgccgtt

SEQ ID NO: 12 - DNA, 21 bp, Artificial Sequence
  Description of Artificial Sequence Synthetic Primer
gtttgcagccatggccttgtt

SEQ ID NO: 13 - DNA, 22 bp, Artificial Sequence
  Description of Artificial Sequence Synthetic Primer
ggcgggaaacgacaatctgatc

SEQ ID NO: 14 - DNA, 32 bp, Artificial Sequence
  Description of Artificial Sequence Synthetic Primer
tatagaattcatggataccgagtttctccgaa

SEQ ID NO: 15 - DNA, 34 bp, Artificial Sequence
  Description of Artificial Sequence Synthetic Primer
tatactgcagtcagaactggaattgcatctccgg

SEQ ID NO: 16 - DNA, 16 bp, Artificial Sequence
  Description of Artificial Sequence Synthetic Primer
agaattcgcggccgct

SEQ ID NO: 17 - DNA, 32 bp, Artificial Sequence
  Description of Artificial Sequence Synthetic Primer
ctcatgcggccgcgcgcaacgcaattaatgtg

SEQ ID NO: 18 - DNA, 32 bp, Artificial Sequence
  Description of Artificial Sequence Synthetic Primer
tcatgcggccgcgagatccagttcgatgtaac

SEQ ID NO: 19 - DNA, 21 bp, Artificial Sequence
  Description of Artificial Sequence Synthetic Primer
gtggattgatgtgatatctcc

SEQ ID NO: 20 - DNA, 21 bp, Artificial Sequence
  Description of Artificial Sequence Synthetic Primer
gtaaggatctgagctacacat

SEQ ID NO: 21 - DNA, 22 bp, Artificial Sequence
  Description of Artificial Sequence Synthetic Primer
atggataccgagtttctccgaa

SEQ ID NO: 22 - DNA, 21 bp, Artificial Sequence
  Description of Artificial Sequence Synthetic Primer
aactggaattgcatctccgga

(19) United States
(12) Patent Application Publication
(10) Pub. No.: US 2006/0160700 A1
(43) Pub. Date: Jul. 20, 2006

(54) Nadh-dependent cytochrome b5 reductase as target for herbicides

(75) Inventor: Andreas Reindl, Mannheim (DE)

Correspondence Address: HUTCHISON LAW GROUP PLLC, PO BOX 31686, RALEIGH, NC, 27612, US

(73) Assignee: Basf Aktiengesellschaft, Ludwigshafen (DE)

(21) Appl. No.: 10/521,584

(22) PCT Filed: Jul. 14, 2003
(86) PCT No.: PCT/EP03/07590

§ 371 Date: Jan. 18, 2005

(30) Foreign Application Priority Data
Jul. 18, 2002 (DE) 10232778.5

(51) Int. Cl. A01N 25/00; C12Q 1/68; C07H 21/04; C12P 21/06; C12N 9/02; C12N 5/04

(52) U.S. Cl. 504/116.1; 435/69.1; 435/189; 435/419; 435/468; 435/6; 536/23.2

(57) ABSTRACT

The present invention relates to the use of a polypeptide with the biological activity of an NADH-dependent cytochrome b5 reductase (E.C. 1.6.2.2), which, when not present, brings about growth retardation symptoms and chlorotic leaves, and which is encoded by the nucleic acid sequence SEQ ID NO:1 or functional equivalents of the abovementioned nucleic acid sequence, as target for herbicides. Functional equivalents of SEQ ID NO:1 are provided in this context. Moreover, the present invention relates to the use of the polypeptide with the biological activity of an NADH-dependent cytochrome b5 reductase in a method for identifying herbicidally active compounds which inhibit NADH-dependent cytochrome b5 reductase. Moreover, the invention relates to the compounds identified by the method for use as herbicides.

The present invention relates to the use of a polypeptide with the biological activity of an NADH-dependent cytochrome b5 reductase (E.C. 1.6.2.2), which, when not present, brings about growth retardation symptoms and chlorotic leaves, and which is encoded by the nucleic acid sequence SEQ ID NO:1 or functional equivalents of the abovementioned nucleic acid sequence, as target for herbicides. Functional equivalents SEQ ID NO:1 are provided in this context. Moreover, the present invention relates to the use of the polypeptide with the biological activity of an NADH-dependent cytochrome b5 reductase in a method for identifying herbicidally active compounds which inhibit NADH-dependent cytochrome b5 reductase. Moreover, the invention relates to the compounds identified by the method for use as herbicides.

The basic principle of identifying herbicides by inhibiting a defined target is known (for example U.S. Pat. No. 5,187,071, WO 98/33925, WO 00/77185). In general, there is a great demand for the detection of enzymes which might constitute novel targets for herbicides. Reasons herefore are that herbicidal active ingredients which act on known targets demonstrate the development of resistance problems, and the constant endeavor to identify novel herbicidal active ingredients which are distinguished by as broad as possible a range of action, ecofriendliness and toxicological compatibility and/or low application rates.

In practice, the detection of novel targets always entails great difficulties since the inhibition of an enzyme which is part of a metabolic pathway frequently has no further effect on the plant's growth. The reason may be that the plant switches over to alternative metabolic pathways whose existence is not known, or that the enzyme which is being inhibited is not limiting for the metabolic pathway. Furthermore, plant genomes are distinguished by a high degree of functional redundance. In the Arabidopsis thaliana genome, functionally equivalent enzymes are more frequently found in gene families than is the case with insects or mammals (Nature, 2000, 408(6814):796-815). This hypothesis is confirmed experimentally by the fact that large gene knock-out programs by the insertion of T-DNA or transposons into Arabidopsis have, as yet, yielded fewer manifested phenotypes than expected (Curr. Op. Plant Biol. 4, 2001, pp. 111-117).

It is an object of the present invention to identify novel targets which are essential for the growth of plants or whose inhibition leads to reduced plant growth, and to provide methods which are suitable for identifying herbicidally active compounds.

We have found that this object is achieved by the use of a polypeptide with the biological activity of an NADH-dependent cytochrome b5 reductase encoded by a nucleic acid sequence consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation; or
d) a functional equivalent of the nucleic acid sequence SEQ ID NO:1 with at least 52% identity with SEQ ID NO:1
   as target for herbicides.

Further terms used in the description are now defined at this point.

“Affinity tag”: This refers to a peptide or polypeptide whose coding nucleic acid sequence can be fused to the nucleic acid sequence according to the invention either directly or by means of a linker, using customary cloning techniques. The affinity tag serves for the isolation, concentration and/or specific purification of the recombinant target protein by means of affinity chromatography from total cell extracts. The abovementioned linker can advantageously contain a protease cleavage site (for example for Thrombin or Factor Xa), whereby the affinity tag can be cleaved from the target protein when required. Examples of customary affinity tags are the “His tag”, for example from Quiagen, Hilden, the “Strep tag”, the “Myc tag” (Invitrogen, Carlsberg), the tag from New England Biolabs which consists of a chitin-binding domain and an intein, the maltose-binding protein (pMal) from New England Biolabs, and what is known as the CBD tag from Novagen. In this context, the affinity tag can be attached to the 5′ or the 3′ end of the coding nucleic acid sequence with the sequence encoding the target protein.

“Nucleic acid sequence according to the invention”: This term is defined further below.

“Expression cassette”: an expression cassette contains a nucleic acid sequence according to the invention linked operably to at least one genetic control element, such as a promoter, and, advantageously, to a further control element, such as a terminator. The nucleic acid sequence of the expression cassette can be, for example, a genomic or complementary DNA sequence or an RNA sequence, and the semi- or fully synthetic analogs thereof. These sequences can exist in linear or circular form, extrachromosomally or integrated into the genome. The nucleic acid sequences in question can be synthesized or obtained naturally or contain a mixture of synthetic and natural DNA components, or else consist of various heterologous gene segments from various organisms.

Artificial nucleic acid sequences are also suitable in this context as long as they make possible the expression, in a cell or an organism, of a polypeptide with the biological activity of an NADH-dependent cytochrome b5 reductase, which polypeptide is encoded by a nucleic acid sequence according to the invention. For example, synthetic nucleotide sequences can be generated which have been optimized with regard to the codon usage of the organisms to be transformed.

All of the abovementioned nucleotide sequences can be generated from the nucleotide units by chemical synthesis in the manner known per se, for example by fragment condensation of individual overlapping complementary nucleotide units of the double helix. Oligonucleotides can be synthesized chemically for example in the manner known per se using the phosphoamidite method (Voet, Voet, 2^{nd} Edition, Wiley Press New York, pp. 896-897). When preparing an expression cassette, various DNA fragments can be manipulated in such a way that a nucleotide sequence with the correct direction of reading and the correct reading frame is obtained. The nucleic acid fragments are linked with each other via general cloning techniques as are described, for example, in T. Maniatis, E. F. Fritsch and J. Sambrook, “Molecular Cloning: A Laboratory Manual”, Cold Spring Harbor Laboratory, Cold Spring Harbor, N.Y. (1989), and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., “Current Protocols in Molecular Biology”, Greene Publishing Assoc. and Wiley-Interscience (1994).

“Operable linkage”: an operable, or functional, linkage is understood as meaning the sequential arrangement of regulatory sequences or genetic control elements in such a way that each of the regulatory sequences, or each of the genetic control elements, can fulfill its intended function when the coding sequence is expressed.

“Functional equivalents” describe, in the present context, nucleic acid sequences which hybridize under standard conditions with the nucleic acid sequence SEQ ID NO:1 or parts of SEQ ID NO:1 and which are capable of bringing about the expression, in a cell or an organism, of a polypeptide with the biological activity of an NADH-dependent cytochrome b5 reductase.

To carry out the hybridization, it is advantageous to use short oligonucleotides with a length of approximately 10-50 bp, preferably 15-40 bp, for example of the conserved or other regions, which can be determined in the manner with which the skilled worker is familiar by comparisons with other related genes. However, longer fragments of the nucleic acids according to the invention with a length of 100-500 bp, or the complete sequences, may also be used for the hybridization. Depending on the nucleic acid/oligonucleotide used, or the length of the fragment or the complete sequence, or on the type of nucleic acid, i.e. DNA or RNA, that is being used for the hybridization, these standard conditions vary. Thus, for example, the melting temperatures for DNA:DNA hybrids are approximately 10° C. lower than those of DNA:RNA hybrids of the same length.

Standard hybridization conditions are to be understood as meaning, depending on the nucleic acid, for example temperatures of between 42 and 58° C. in an aqueous buffer solution with a concentration of between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. and 65° C., preferably between approximately 30° C. and 45° C. In the case of DNA:RNA hybrids, the hybridization conditions are advantageously 0.1×SSC and temperatures of between approximately 30° C. and 65° C., preferably between approximately 45° C. and 55° C. These hybridization temperatures which have been stated are melting temperature values which have been calculated by way of example for a nucleic acid with a length of approx. 100 nucleotides and a G+C content of 50% in the absence of formamide. The experimental conditions for DNA hybridization are described in specialist textbooks of genetics such as, for example, in Sambrook et al., “Molecular Cloning”, Cold Spring Harbor Laboratory, 1989, and can be calculated using formulae with which the skilled worker is familiar, for example as a function of the length of the nucleic acids, the type of the hybrids or the G+C content. The skilled worker will find further information on hybridization in the following text books: Ausubel et al. (eds), 1985, “Current Protocols in Molecular Biology”, John Wiley & Sons, New York; Hames and Higgins (eds), 1985, “Nucleic Acids Hybridization: A Practical Approach”, IRL Press at Oxford University Press, Oxford; Brown (ed), 1991, Essential Molecular Biology: A Practical Approach, IRL Press at Oxford University Press, Oxford.

A functional equivalent of the SEQ ID NO:1 is furthermore also understood as meaning nucleic acid sequences which have up to a defined degree of homology or identity with SEQ ID NO:1, and furthermore in particular also natural or artificial mutations of the abovementioned nucleic acid sequences which encode a polypeptide with the biological activity of an NADH-dependent cytochrome b5 reductase.

For example, the present invention also encompasses, for example, those nucleotide sequences which are obtained by modification of the abovementioned nucleic acid sequences. For example, such modifications can be generated by techniques with which the skilled worker is familiar, such as “site directed mutagenesis”, “error prone PCR”, “DNA-shuffling” (Nature 370, 1994, pp. 389-391) or “staggered extension process” (Nature Biotechnol. 16, 1998, pp. 258-261). The purpose of such a modification can be, for example, the insertion of further cleavage sites for restriction enzymes, the removal of DNA in order to truncate the sequence, the substitution of nucleotides to optimize the codons, or the addition of further sequences. Proteins which are encoded via modified nucleic acid sequences must retain the desired functions despite a deviating nucleic acid sequence.

The term “functional equivalent” can also relate to the amino acid sequence encoded by the nucleic acid sequence in question. In this case, the term “functional equivalent” describes a protein whose amino acid sequence has up to a defined percentage identity or homology with SEQ ID NO:2.

Functional equivalents thus comprise naturally occurring variants of the herein-described sequences and also artificial nucleic acid sequences, for example those which have been obtained by chemical synthesis and which are adapted to the codon usage, and also the amino acid sequences derived from them.

“Genetic control sequence” describes sequences which have an effect on the transcription and, if appropriate, translation of the nucleic acids according to the invention in prokaryotic or eukaryotic organisms. Examples are promoters, terminators or what are known as “enhancer” sequences. In addition to these control sequences, or instead of these sequences, the natural regulation of these sequences may still be present before the actual structural genes and may, if appropriate, have been genetically modified in such a way that the natural regulation has been switched off and the expression of the target gene has been modified, that is to say increased or reduced. The choice of the control sequence depends on the host organism or the starting organism. Genetic control sequences furthermore also comprise the 5′-untranslated region, introns or the noncoding 3′ region of genes. Control sequences are furthermore understood as meaning those which make possible homologous recombination or insertion into the genome of a host organism, or which permit removal from the genome. Genetic control sequences also comprise further promoters, promoter elements or minimal promoters, and sequences which have an effect on the chromatin structure (for example matrix attachment regions (MARs)), which can modify the expression-governing properties. Thus, genetic control sequences may bring about, for example, the fact that the tissue-specific expression is additionally dependent on certain stress factors. Such elements have been described, for example, for water stress, abscisic acid (Lam E and Chua N H, J Biol Chem 1991; 266(26): 17131-17135), low temperature stress, drought stress (Plant Cell 1994, (6): 251-264) and high temperature stress (Molecular & General Genetics, 1989, 217(2-3): 246-53).

“Homology” between two nucleic acid sequences or polypeptide sequences is defined by the identity of the nucleic acid sequence/polypeptide sequence over in each case the entire length of the sequence of the shorter sequence of the two, and this identity is calculated by alignment with the aid of the program algorithm GAP (Wisconsin Package Version 10.2 Genetics Computer Group (GCG), Madison, Wis., USA), setting the following parameters for polypeptides

| | |
|---|---|
| Gap Weight: 8 | Length Weight: 2 |
| Average Match: 2.912 | Average Mismatch: −2.003 |

and the following parameters for nucleic acids:

| | |
|---|---|
| Gap Weight: 50 | Length Weight: 3 |
| Average Match: 10.000 | Average Mismatch: −0.000 |

In the following text, the term “identity” is also used synonymously with the term “homologous” or “homology”.

“Mutations” of nucleic or amino acid sequences encompass substitutions, additions, deletions, inversions or insertions of one or more nucleotide residues, which may also bring about changes in the corresponding amino acid sequence of the target protein by substitution, insertion or deletion of one or more amino acids, but where the functional properties of the target protein in total are essentially retained.

“Natural genetic environment” refers to the natural chromosomal locus in the organism of origin. In the case of a genomic library, the natural genetic environment of the nucleic acid sequence is preferably retained at least in part. The environment flanks the nucleic acid sequence at least at 5′ or 3′ and has a sequence length of at least 50 bp, preferably at least 100 bp, especially preferably at least 500 bp, very especially preferably at least 1000 bp, most preferably at least 5000 bp.

“Plants” for the purposes of the invention are plant cells, plant tissues, plant organs, or intact plants, such as seeds, tubers, flowers, pollen, fruits, seedlings, roots, leaves, stems or other plant parts. Moreover, the term plants is understood as meaning propagation material such as seeds, fruits, seedlings, slips, tubers, cuttings or root stocks.

“Reaction time” refers to the time required for carrying out an activity assay until a significant finding regarding an activity is obtained; it depends both on the specific activity of the protein employed in the assay and on the method used and the sensitivity of the apparatus used. The skilled worker is familiar with the determination of the reaction times. In the case of methods for identifying herbicidally active compounds which are based on photometry, the reaction times are, for example, between >0 and 120 minutes.

“Recombinant DNA” describes a combination of DNA sequences which can be generated by recombinant DNA technology.

“Recombinant DNA technology”: generally known techniques for fusing DNA sequences (for example described in Sambrook et al., 1989, Cold Spring Habour, N.Y., Cold Spring Habour Laboratory Press).

“Replication origins” ensure the replication of the expression cassettes or vectors according to the invention in microorganisms and yeasts, for example the pBR322 ori or the P15A ori in E. coli (Sambrook et al.: “Molecular Cloning. A Laboratory Manual”, 2nd ed. Cold Spring Harbor Laboratory Press, Cold Spring Harbor, N.Y., 1989) and the ARS1 ori in yeast (Nucleic Acids Research, 2000, 28(10): 2060-2068).

“Reporter genes” encode readily quantifiable proteins. The transformation efficacy or the transformation site or timing can be assessed by means of these genes via a growth assay, fluorescence assay, chemoluminescence assay, bioluminescence assay or resistance assay or via a photometric measurement (intrinsic color) or enzyme activity. Very especially preferred in this context are reporter proteins (Schenborn E, Groskreutz D. Mol Biotechnol. 1999; 13(1):29-44) such as the “green fluorescence protein” (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol acetyltransferase, a luciferase (Giacomin, Plant Sci 1996, 116:59-72; Scikantha, J Bact 1996, 178:121; Millar et al., Plant Mol Biol Rep 1992 10:324-414), and luciferase genes, in general β-galactosidase or β-glucuronidase (Jefferson et al., EMBO J. 1987, 6, 3901-3907) or the Ura3 gene.

“Selection markers” confer a resistance to antibiotics or other toxic compounds: examples which may be mentioned in this context are the neomycin phosphotransferase gene, which confers resistance to the aminoglycoside antibiotics neomycin (G 418), kanamycin, paromycin (Deshayes A et al., EMBO J. 4 (1985) 2731-2737), the sul gene encoding a mutated dihydropteroate synthase (Guerineau F et al., Plant Mol Biol. 1990; 15(1):127-136), the hygromycin B phosphotransferase gene (Gen Bank Accession NO: K 01193) and the shble resistance gene, which confers resistance to the bleomycin antibiotics such as, for example, zeocin. Further examples of selection marker genes are genes which confer resistance to 2-deoxyglucose-6-phosphate (WO 98/45456) or phosphinothricin and the like, or those which confer a resistance to antimetabolites, for example the dhfr gene (Reiss, Plant Physiol. (Life Sci. Adv.) 13 (1994) 142-149). Examples of other genes which are suitable are trpB or hisD (Hartman S C and Mulligan R C, Proc Natl Acad Sci USA. 85 (1988) 8047-8051). Another suitable gene is the mannose-phosphate isomerase gene (WO 94/20627), the ODC (ornithin decarboxylase) gene (McConlogue, 1987 in: Current Communications in Molecular Biology, Cold Spring Harbor Laboratory, Hrsg.) or the Aspergillus terreus deaminase (Tamura K et al., Biosci Biotechnol Biochem. 59 (1995) 2336-2338).

“Transformation” describes a process for introducing heterologous DNA into a prokaryotic or eukaryotic cell. The term transformed cell describes not only the product of the transformation process per se, but also all of the transgenic progeny of the transgenic organism generated by the transformation.

“Target/target protein”: a polypeptide encoded by the nucleic acid sequence according to the invention, which may take the form of an enzyme in the traditional sense or, for example, of a structural protein, development protein, regulatory proteins such as transcription factors, kinases, phosphatases, receptors, channel subunits, transport proteins, regulatory subunits which confer substrate or activity regulation to an enzyme complex. All of the targets or sites of action share the characteristic that their functional presence is essential for the survival or the normal development and growth.

“Transgenic”: referring to a nucleic acid sequence, an expression cassette or a vector comprising a nucleic acid sequence according to the invention or an organism transformed with the abovementioned nucleic acid sequence, expression cassette or vector, the term transgenic describes all those constructs which have been generated by recombinant methods in which either the nucleic acid sequence of the target protein or a genetic control sequence linked operably to the nucleic acid sequence of the target protein or a combination of the abovementioned possibilities are not in their natural genetic environment or have been modified by recombinant methods. In this context, the modification can be achieved, for example, by mutating one or more nucleotide residues of the nucleic acid sequence in question.

The nucleic acid sequence SEQ ID NO:1 encodes a specifically NADH-dependent cytochrome b5 reductase (E.C. 1.6.2.2).

The electron transfer system located at the membrane of the endoplasmic reticulum, which is characteristic of higher eukaryotes, consists of an NADH-dependent cytochrome b5 reductase and cytochrome b5 (Cytb5). The NADH-dependent cytochrome b5 reductase, in which FAD is the prosthetic group, transfers electrons from NADH to Cytb5, a heme-containing protein. Thus, polypeptide with the biological activity of an NADH-dependent cytochrome b5 reductase refers to an enzyme which is capable, with an FAD as prosthetic group, of transferring electrons from NADH to Cytb5, a heme-containing protein. The enzymatic activity of an enzyme with the biological activity of an NADH-dependent cytochrome b5 reductase can be determined by means of suitable activity assays as are described by way of example further below (see also Example 5, inter alia).

In plants, Cytb5 was described as component of the electron transport in the modification of fatty acids (Kearns et al., 1991; Spektrum Akademischer Verlag Heidelberg, Berlin, Oxford, pp. 751). It is assumed that the electrons are subsequently transferred from cytochrome b5 to desaturases or P450 monooxygenases (Fukuchi-Mizutani, Plant Physiology, 119; 353-361; 1999). Plant NADH-dependent cytochrome b5 reductases are found in virtually all cell types, in particular in immature seeds (Fukuchi-Mizutani, Plant Physiology, 119; 353-361; 1999).

The NADH-dependent cytochrome b5 reductase compound was first isolated and characterized from human erythrocytes (Yubisui T, Takeshita M., J Biol Chem., 1980;255(6):2454-1456) and since then from a large number of other organisms. Nucleic acid sequences of plant NADH-dependent cytochrome b5 reductases are known from, for example, Arabidopsis (Gen Bank Acc. No. AB007799; Mizutani and Fukuchi-Mizutani, Plant Physiol. 119, 353-361; 1999) and ESTs of plant NADH-dependent cytochrome b5 reductases from Medicago truncatula (Gen Bank Acc. No. AA660929; Covitz, P. A. et al. Plant Physiol. 117 (4), 1325-1332 (1998) Identity with SEQ ID NO:1=68.763%, Identity with SEQ ID NO:2=46.897%), Oryza sativa (Gen Bank Acc. No. BE039960; Identity with SEQ ID NO:1=69.457%, Identity with SEQ ID NO:2=75.912%), Solanum tuberosum (Gen Bank Acc. No. BE340917, Identity with SEQ ID NO:1=75.564%, Identity with SEQ ID NO:2=81.675%) and Beta vulgaris (Gen Bank Acc. No. BI096337; Identity with SEQ ID NO:1=52.727%; Identity with SEQ ID NO:2=39.552%) and squash (Cucurbita maxima; Gen Bank Acc. No. AF274589; Identity with SEQ ID NO:1=56.703%, Identity with SEQ ID NO:2=43.621%).

The NADH-dependent cytochrome b5 reductase from human erthyrocytes can be inhibited by inositol hexaphosphate concentrations in the millimolar range (Palmieri et al., Archives of Biochemistry and Biophysics, 1990, 280(1), 224-228). At a concentration of 0.5 mM, thenoyltrifluoroacetone shows 50% inhibition of NADH-dependent cytochrome b5 reductase from rat liver microsomes (Golf et al., Biol. Chem. Hoppe-Seyler 1985, 366, 647-653). Other substances which are capable of inhibiting NADH-dependent cytochrome b5 reductase from various organisms are amytal, mepacrin, dicoumarol (Golf et al.; 1985, Biol. Chem. Hoppe-Seyler, 366, pp. 647-653), or N-ethylmaleimide and atebrin (Tamura et al.; 1983, J. Biochem. 94, pp. 1547-1555). However, inhibitors for plant NADH-dependent cytochrome b5 reductases have not been described to date.

Surprisingly, it has been found within the scope of the present invention that plants in which the activity of NADH-dependent cytochrome b5 reductase was reduced in a targeted fashion displayed phenotypes which are comparable with phenotypes generated by the application of herbicide. Among the symptoms observed were growth retardation and necrotic, stressed leaves and, in some cases, the death of entire plants, or of plant parts. The pods of these plants were either empty or contained shriveled seeds, none of which was capable of germination.

The present invention relates to the use of a polypeptide with the biological activity of an NADH-dependent cytochrome b5 reductase encoded by a nucleic acid sequence consisting of
a) a nucleic acid sequence with the nucleic acid sequence shown in SEQ ID NO:1; or;
b) a nucleic acid sequence which, on the basis of the degeneracy of the genetic code, can be deduced from the amino acid sequence shown in SEQ ID NO:2 by back translation; or
c) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation; or
d) a functional equivalent of the nucleic acid sequence SEQ ID NO:1 with at least 52% identity with SEQ ID NO:1;
   as target for herbicides. The functional equivalents of c) are distinguished by an essentially identical functionality, i.e. they have the physiological function of an NADH-dependent cytochrome b5 reductase.

The functional equivalents according to the invention of SEQ ID NO:1 have at least 52%, 53%, 54%, 55%, 56%, 57%, 58%, 59%, by reference at least 60%, 61%, 62%, 63%, 64%, 65%, 66%, 67%, 68%, 69% and 70%, preferably at least 71%, 72%, 73%, 74%, 75%, 76%, especially preferably at least 77%, 78%, 79%, 80%, 81%, 82%, 83%, 84%, 85%, 86%, 87%, 88%, 89%, 90%, very especially preferably at least 91%, 92%, 93%, 94%, 95%, 96%, 97%, 98%, 99% homology with SEQ ID NO:1.

The functional equivalents according to the invention of SEQ ID NO:2 have at least 39%, 40%, 41%, 42%, 43%, 44%, 45%, 46%, 47%, 48%, 49%, 50%, 51%, 52%, 53%, 54%, 55%, 56%, 57%, 58%, 59%, by preference at least 60%, 61%, 62%, 63%, 64%, 65%, 66%, 67%, 68%, 69% and 70%, preferably at least 71%, 72%, 73%, 74%, 75%, 76%, 77%, 78%, 79%, 80%, 81%, 82%, 83%, 84%, 85%, 86%, especially preferably at least 87%, 88%, 89%, 90%, 91%, 92%, 93%, very especially preferably at least 94%, 95%, 96%, 97%, 98%, 99% homology with SEQ ID NO:2.

Examples of functional equivalents are the plant nucleic acid sequences which encode NADH-dependent cytochrome b5 reductase and which have already been mentioned further above or amino acid sequences of an NADH-dependent cytochrome b5 reductase from Medicago truncatula (Gen Bank Acc. No. AA660929; Covitz, P. A. et al. Plant Physiol. 117 (4), 1325-1332 (1998)), Oryza sativa (Gen Bank Acc. No. BE039960), Solanum tuberosum (Gen Bank Acc. No. BE340917), Beta vulgaris (Gen Bank Acc. No. BI096337) and squash (Cucurbita maxima; Gen Bank Acc. No. AF274589).

Moreover, the present invention claims functional equivalents of the abovementioned nucleic acid sequences which encode a polypeptide with the biological activity of an NADH-dependent cytochrome b5 reductase comprising a part-region encompassing:
a) a nucleic acid sequence with the nucleic acid sequence shown in SEQ ID NO:3; or
b) a nucleic acid sequence which, on the basis of the degeneracy of the genetic code, can be deduced from the amino acid sequence shown in SEQ ID NO:4 by back translation; or
c) functional equivalents of the nucleic acid sequence SEQ ID NO:3 with at least 77% identity with SEQ ID NO:3; or
d) a nucleic acid sequence which, on the basis of the degeneracy of the genetic code, can be deduced from the amino acid sequence of a functional equivalent of SEQ ID NO:4, which has at least 87% identity with SEQ ID NO:4; by back translation.

The polypeptides encoded by the abovementioned nucleic acid sequences are likewise claimed. The functional equivalents are distinguished by an essentially identical functionality, i.e. they have the physiological function of an NADH-dependent cytochrome b5 reductase.

The term “encompassing” or “to encompass” in the context of nucleic acid sequences refers to the fact that the nucleic acid sequence according to the invention may contain additional nucleic acid sequences at the 3′ or 5′ end, the length of the additional nucleic acid sequences not exceeding 75 bp of the 5′ end and 50 bp of the 3′ end, preferably 50 bp at the 5′ end and 10 bp at the 3′ end, of the nucleic acid sequences according to the invention.

The functional equivalents according to the invention of SEQ ID NO:3 have at least 77%, 78%, 79%, 80%, preferably at least 81%, 82%, 83%, 84%, 85%, 86%, 87%, 88%, 89%, 90%, especially preferably at least 91%, 92%, 93%, 94%, 95%, 96%, 97%, 98%, 99% homology with SEQ ID NO:3.

The functional equivalents according to the invention of SEQ ID NO:4 have at least 87%, by preference at least 88%, 89%, preferably at least 90%, 91%, 92%, 93%, especially preferably at least 94%, 95%, 96%, very especially preferably at least 97%, 98%, 99% homology with SEQ ID NO:4.

Nucleic acid sequences encoding a polypeptide with the biological activity of an NADH-dependent cytochrome b5 reductase consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation; or
d) a functional equivalent of the nucleic acid sequence SEQ ID NO:1 with at least 52% identity with SEQ ID NO:1; or
   nucleic acid sequences encoding a polypeptide with the biological activity of an NADH-dependent cytochrome b5 reductase comprising a part-region encompassing:
a) a nucleic acid sequence with the nucleic acid sequence shown in SEQ ID NO:3; or
b) a nucleic acid sequence which, on the basis of the degeneracy of the genetic code, can be deduced from the amino acid sequence shown in SEQ ID NO:4 by back translation; or
c) functional equivalents of the nucleic acid sequence SEQ ID NO:3 with at least 86% identity with SEQ ID NO:3; or
d) a nucleic acid sequence which, on the basis of the degeneracy of the genetic code, can be deduced from the amino acid sequence of a functional equivalent of SEQ ID NO:4, which has at least 87% identity with SEQ ID NO:4, by back translation;
   are hereinbelow referred to as “nucleic acid sequences according to the invention. The polypeptides encoded by a nucleic acid sequence according to the invention with the biological activity of an NADH-dependent cytochrome b5 reductase are hereinbelow referred to as “NCRs” for the sake of simplicity.

NCRs, in reduced quantity, cause growth retardation symptoms and necrotic leaves in plants. A reduction in the polypeptide means that the amount of the polypeptide is reduced by recombinant methods. A plant which has been modified thus is compared with a plant which has not been genetically modified with regard to this polypeptide, but which is otherwise identical with the genotype of the genetically manipulated plant under identical growth conditions.

The gene products of the nucleic acids according to the invention constitute novel targets for herbicides which allow novel herbicides for controlling undesired plants to be provided.

Undesired plants are understood as meaning, in the broadest sense, all those plants which grow at locations where they are undesired, for example:

Dicotyledonous weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds from the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

SEQ ID NO:1 or SEQ ID NO:3 or parts of the abovementioned nucleic acid sequences can be used for the preparation of hybridization probes, by means of which for example the corresponding full-length genes and/or functional equivalents of SEQ ID NO:1 or SEQ ID NO:3 can be isolated. The preparation of these probes and the experimental procedure is known. For example, this can be effected via the tailor-made preparation of radioactive or nonradioactive probes by means of PCR and the use of suitably labeled oligonucleotides, followed by hybridization experiments. The technologies required for this purpose are given, for example, in T. Maniatis, E. F. Fritsch and J. Sambrook, “Molecular Cloning: A Laboratory Manual”, Cold Spring Harbor Laboratory, Cold Spring Harbor, N.Y. (1989). The probes in question can furthermore be modified by standard technologies (Lit. SDM or random mutagenesis) so that they can be employed for further purposes, for example as probe which hybridizes specifically with mRNA and the corresponding coding sequences, in order to analyze the corresponding sequences in other organisms.

Moreover, the abovementioned probes can be used for the detection and isolation of functional equivalents of SEQ ID NO:1 or SEQ ID NO:3 from other plant species on the basis of sequence identities. In this context, part or all of the sequence of corresponding SEQ ID NO:1 or SEQ ID NO:3 is used as probe for screening in a genomic library or cDNA library of the plant species in question or in a computer search for sequences of functional equivalents in electronic databases.

Preferred plant species are the undesired plants which have already been mentioned at the outset.

The invention furthermore relates to expression cassettes comprising
a) genetic control sequences in functional linkage with a nucleic acid sequence encompassing a part-region comprising a nucleic acid sequence with the nucleic acid sequence shown in SEQ ID NO:3; or a nucleic acid sequence which, on the basis of the degeneracy of the genetic code, can be deduced from the amino acid sequence shown in SEQ ID NO:4 by back translation; or functional equivalents of the nucleic acid sequence SEQ ID NO:3 with at least 86% identity with SEQ ID NO:3; a nucleic acid sequence which, on the basis of the degeneracy of the genetic code, can be deduced from the amino acid sequence of the functional equivalent of SEQ ID NO:4, which has at least 87% identity with SEQ ID NO:4, by back translation;
b) additional functional elements; or
c) a combination of a) and b);
   and to the use of expression cassettes comprising
a) genetic control sequences in operable linkage with a nucleic acid sequence according to the invention;
b) additional functional elements; or
c) a combination of a) and b);
   for expressing an NCR which can be used in “in vitro” assay systems. Both embodiments of the above-described expression cassettes are hereinbelow referred to as expression cassettes according to the invention.

In a preferred embodiment, an expression cassette according to the invention comprises a promoter at the 5′ end of the coding sequence and, at the 3′ end, a transcription termination signal and, if appropriate, further genetic control sequences which are linked operably with the interposed nucleic acid sequence according to the invention.

The expression cassettes according to the invention are also understood as meaning analogs which can be brought about, for example, by a combination of individual nucleic acid sequences on a polynucleotide (multiple constructs, on a plurality of polynucleotides in a cell (cotransformation) or by sequential transformation.

Advantageous genetic control sequences under item a) for the expression cassettes according to the invention or for vectors comprising expression cassettes according to the invention are, for example, promoters such as cos, tac, trp, tet, lpp, lac, laciq, T7, T5, T3, gal, trc, ara, SP6, λ-PR or the λ-PL promoter, all of which can be used for expressing NCR in Gram-negative bacterial strains.

Examples of further advantageous genetic control sequences are present, for example, in the promoters amy and SPO2, both of which can be used for expressing NCR in Gram-positive bacterial strains, and in the yeast or fungal promoters AUG1, GPD-1, PX6, TEF, CUP1, PGK, GAP1, TPI, PHO5, AOX1, GAL10/CYC1, CYC1, OliC, ADH, TDH, Kex2, MFa or NMT or combinations of the abovementioned promoters (Degryse et al., Yeast 1995 Jun. 15; 11(7):629-40; Romanos et al. Yeast 1992 June;8(6):423-88; Benito et al. Eur. J. Plant Pathol. 104, 207-220 (1998); Cregg et al. Biotechnology (N.Y.) 1993 August;11(8):905-10; Luo X., Gene 1995 Sep. 22;163(1):127-31; Nacken et al., Gene 1996 Oct. 10;175(1-2): 253-60; Turgeon et al., Mol Cell Biol 1987 September;7(9):3297-305) or the transcription terminators NMT, Gcy1, TrpC, AOX1, nos, PGK or CYC1 (Degryse et al., Yeast 1995 Jun. 15; 11(7):629-40; Brunelli et al. Yeast 1993 Dec. 9(12): 1309-18; Frisch et al., Plant Mol. Biol. 27 (2), 405-409 (1995); Scorer et al., Biotechnology (N.Y.) 12 (2), 181-184 (1994), Genbank Acc. number Z46232; Zhao et al. Genbank Acc number: AF049064; Punt et al., (1987) Gene 56 (1), 117-124), all of which can be used for expressing NCR in yeast strains.

Examples of genetic control sequences which are suitable for expression in insect cells are the polyhedrin promoter and the p10 promoter (Luckow, V. A. and Summers, M. D. (1988) Bio/Techn. 6, 47-55).

Advantageous genetic control sequences for expressing NCR in cell culture are, in addition to polyadenylation sequences such as, for example, from simian virus 40, eukaryotic promoters of viral origin such as, for example, promoters of the polyoma virus, adenovirus 2, cytomegalovirus or simian virus 40.

Further advantageous genetic control sequences for expressing NCR in plants are present in the plant promoters CaMV/35S [Franck et al., Cell 21(1980) 285-294], PRP1 [Ward et al., Plant. Mol. Biol. 22 (1993)], SSU, OCS, LEB4, USP, STLS1, B33, NOS; FBPaseP (WO 98/18940) or in the ubiquitin or phaseolin promoter; a promoter which is preferably used being, in particular, a plant promoter or a promoter derived from a plant virus. Especially preferred are promoters of viral origin, such as the promoter of the cauliflower mosaic virus 35S transcript (Franck et al., Cell 21 (1980), 285-294; Odell et al., Nature 313 (1985), 810-812). Further preferred constitutive promoters are, for example, the Agrobacterium nopaline synthase promoter, the TR dual promoter, the Agrobacterium OCS (octopine synthase) promoter, the ubiquitin promoter (Holtorf S et al., Plant Mol Biol 1995, 29:637-649), the promoters of the vacuolar ATPase subunits, or the promoter of a proline-rich protein from wheat (WO 91/13991).

The expression cassettes may also comprise, as genetic control sequence, a chemically inducible promoter, by means of which the expression of the exogenous gene in the plant can be controlled at a specific point in time. Such promoters, such as, for example, the PRP1 promoter (Ward et al., Plant. Mol. Biol. 22 (1993), 361-366), a salicylic-acid-inducible promoter (WO 95/19443), a benzenesulfonamide-inducible promoter (EP-A-0388186), a tetracyclin-inducible promoter (Gatz et al., (1992) Plant J. 2, 397404), an abscisic-acid-inducible promoter (EP-A 335528) or an ethanol- or cyclohexanone-inducible promoter (WO 93/21334) may also be used.

Furthermore, suitable promoters are those which confer tissue- or organ-specific expression in, for example, anthers, ovaries, inflorescences and floral organs, leaves, stomata, trichomes, stems, vascular tissues, roots and seeds. Others which are suitable in addition to the abovementioned constitutive promoters are, in particular, those promoters which ensure leaf-specific expression. Promoters which must be mentioned are the potato cytosolic FBPase promoter (WO 97/05900), the rubisco (ribulose-1,5-bisphosphate carboxylase) SSU (small subunit) promoter or the ST-LSI promoter from potato (Stockhaus et al., EMBO J. 8 (1989), 2445-245). Promoters which are furthermore preferred are those which control expression in seeds and plant embryos. Examples of seed-specific promoters are the phaseolin promoter (U.S. Pat. No. 5,504,200, Bustos M M et al., Plant Cell. 1989;1(9):839-53), the promoter of the 2S albumin gene (Joseffson L G et al., J Biol Chem 1987, 262:12196-12201), the legumin promoter (Shirsat A et al., Mol Gen Genet. 1989;215(2):326-331), the USP (unknown seed protein) promoter (Baumlein H et al., Molecular & General Genetics 1991, 225(3):459-67), the napin gene promoter (Stalberg K, et al., L. Planta 1996, 199:515-519), the sucrose binding protein promoter (WO 00/26388) or the LeB4 promoter (Baumlein H et al., Mol Gen Genet 1991, 225: 121-128; Fiedler, U. et al., Biotechnology (NY) (1995), 13 (10) 1090).

Further promoters which are suitable as genetic control sequences are, for example, specific promoters for tubers, storage roots or roots, such as, for example, the class I patatin promoter (B33), the potato cathepsin D inhibitor promoter, the starch synthase (GBSS1) promoter or the sporamin promoter, fruit-specific promoters such as, for example, the fruit-specific promoter from tomato (EP-A 409625), fruit-maturation-specific promoters such as, for example, the fruit-maturation-specific promoter from tomato (WO 94/21794), inflorescence-specific promoters such as, for example, the phytoene synthase promoter (WO 92/16635) or the promoter of the P-rr gene (WO 98/22593), or specific plastid or chromoplast promoters such as, for example, the RNA polymerase promoter (WO 97/06250), or else the Glycine max phosphoribosyl-pyrophosphate amidotransferase promoter (see also Genbank Accession No. U87999), or another node-specific promoter as described in EP-A 249676 may be used advantageously.

Additional functional elements b) are understood as meaning by way of example but not by limitation reporter genes, replication origins, selection markers and what are known as affinity tags, in fusion with NCR directly or by means of a linker optionally comprising a protease cleavage site. Further suitable additional functional elements are sequences which ensure that the product is targeted into the apoplasts, into plastids, the vacuoles, the mitochondrion, the peroxisome, the endoplasmic reticulum (ER) or, owing to the absence of such operative sequences, remains in the compartment where it is formed, the cytosol (Kermode, Crit. Rev. Plant Sci. 15, 4 (1996), 285-423).

Also in accordance with the invention are vectors comprising at least one copy of the nucleic acid sequences according to the invention and/or the expression cassettes according to the invention.

In addition to plasmids, vectors are furthermore also understood as meaning all of the other known vectors with which the skilled worker is familiar, such as, for example, phages, viruses such as SV40, CMV, baculovirus, adenovirus, transposons, IS elements, phasmids, phagemids, cosmids or linear or circular DNA. These vectors can replicate autonomously in the host organism or replicate chromosomally; chromosomal replication is preferred.

In a further embodiment of the vector, the nucleic acid construct according to the invention can advantageously also be introduced into the organisms in the form of a linear DNA and integrated into the genome of the host organism via heterologous or homologous recombination. This linear DNA may consist of a linearized plasmid or only of the nucleic acid construct as vector, or the nucleic acid sequences used.

Further prokaryotic or eukaryotic expression systems are mentioned in Chapters 16 and 17 in Sambrook et al., “Molecular Cloning: A Laboratory Manual.” 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The expression cassette according to the invention and vectors derived therefrom can be used for transforming bacteria, cyanobacteria, yeasts, filamentous fungi and algae and eukaryotic nonhuman cells (for example insect cells) with the aim of producing NCR recombinantly, the generation of a suitable expression cassette depending on the organism in which the gene is to be expressed.

In a further advantageous embodiment, the nucleic acid sequences used in the method according to the invention may also be introduced into an organism by themselves.

If, in addition to the nucleic acid sequences, further genes are to be introduced into the organism, they can all be introduced into the organism together in a single vector, or each individual gene can be introduced into the organism in in each case one vector, it being possible to introduce the different vectors simultaneously or in succession.

In this context, the introduction, into the organisms in question (transformation), of the nucleic acid(s) according to the invention, of the expression cassette or of the vector can be effected in principle by all methods with which the skilled worker is familiar.

In the case of microorganisms, the skilled worker will find suitable methods in the textbooks by Sambrook, J. et al. (1989) “Molecular cloning: A laboratory manual”, Cold Spring Harbor Laboratory Press, by F. M. Ausubel et al. (1994) “Current protocols in molecular biology”, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Harbor Laboratory Press or Guthrie et al. “Guide to Yeast Genetics and Molecular Biology”, Methods in Enzymology, 1994, Academic Press.

In the case of dicots, the methods which have been described for the transformation and regeneration of plants from plant tissues or plant cells can be exploited for transient or stable transformation. Suitable methods are the biolistic method or the transformation of protoplasts (cf., for example, Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge), electroporation, the incubation of dry embryos in DNA-containing solution, microinjection and the agrobacterium-mediated gene transfer. The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993) 128-143 and in Potrykus, Annu. Rev. Plant Physiol. Plant Molec. Biol. 42 (1991) 205-225).

The transformation by means of agrobacteria, and the vectors to be used for the transformation, are known to the skilled worker and described extensively in the literature (Bevan et al., Nucl. Acids Res. 12 (1984) 8711. The intermediary vectors can be integrated into the agrobacterial Ti or Ri plasmid by means of homologous recombination owing to sequences which are homologous to sequences in the T-DNA. This plasmid additionally contains the vir region, which is required for the transfer of the T-DNA. Intermediary vectors are not capable of replication in agrobacteria. The intermediary vector can be transferred to Agrobacterium tumefaciens by means of a helper plasmid (conjugation). Binary vectors are capable of replication both in E. coli and in agrobacteria. They contain a selection marker gene and a linker or polylinker which are framed by the right and left T-DNA border region. They can be transformed directly into the agrobacteria (Holsters et al. Mol. Gen. Genet. 163 (1978), 181-187), EP A 0 120 516; Hoekema, In: The Binary Plant Vector System Offsetdrukkerij Kanters B. V., Alblasserdam (1985), Chapter V; Fraley et al., Crit. Rev. Plant. Sci., 4: 1-46 and An et al. EMBO J. 4 (1985), 277-287).

The transformation of monocots by means of vectors based on Agrobacterium has also been described (Chan et al., Plant Mol. Biol. 22(1993), 491-506; Hiei et al., Plant J. 6 (1994) 271-282; Deng et al.; Science in China 33 (1990), 28-34; wilmink et al., Plant Cell Reports 11, (1992) 76-80; May et al.; Biotechnology 13 (1995) 486-492; Conner and Domisse; Int. J. Plant Sci. 153 (1992) 550-555; Ritchie et al.; Transgenic Res. (1993) 252-265). Alternative systems for the transformation of monocots are the transformation by means of the biolistic approach (Wan and Lemaux; Plant Physiol. 104 (1994), 37-48; Vasil et al.; Biotechnology 11 (1992), 667-674; Ritala et al., Plant Mol. Biol 24, (1994) 317-325; Spencer et al., Theor. Appl. Genet. 79 (1990), 625-631), protoplast transformation, the electroporation of partially permeabilized cells, and the introduction of DNA by means of glass fibers. In particular the transformation of maize has been described repeatedly in the literature (WO 95/06128; EP 0513849 A1; EP 0465875 A1; EP 0292435 A1; Fromm et al., Biotechnology 8 (1990), 833-844; Gordon-Kamm et al., Plant Cell 2 (1990), 603-618; Koziel et al., Biotechnology 11(1993) 194-200; Moroc et al., Theor Applied Genetics 80 (190) 721-726). The generation of protoplasts and transformation with the aid of PEG (Wiebe et al. (1997) Mycol. Res. 101 (7): 971-877; Proctor et al. (1997) Microbiol. 143, 2538-2591), on the one hand, and transformation with the assistance of Agrobacterium tumefaciens (de Groot et al. (1998) Nat. Biotech. 16, 839-842), on the other hand, lend themselves to the transformation of filamentous fungi.

The successful transformation of other cereal species has also already been described for example in the case of barley (Wan and Lemaux, see above; Ritala et al., see above, and wheat (Nehra et al., Plant J. 5(1994) 285-297).

Agrobacteria which have been transformed with a vector according to the invention can likewise be used in a known manner for the transformation of plants, such as test plants like Arabidopsis or crop plants like cereals, maize, oats, rye, barley, wheat, soya, rice, cotton, sugarbeet, canola, sunflower, flax, hemp, potato, tobacco, tomato, carrot, capsicum, oilseed rape, tapioca, cassaya, arrowroot, Tagetes, alfalfa, lettuce and the various tree, nut and grapevine species, for example by bathing scarified leaves or leaf segments in an agrobacterial solution and subsequently growing them in suitable media.

The genetically modified plant cells can be regenerated via all methods with which the skilled worker is familiar. Such methods can be found in the abovementioned publications by S. D. Kung and R. Wu, Potrykus or Höfgen and Willmitzer.

The transgenic organisms generated by transformation with one of the above-described embodiments of an expression cassette comprising a nucleic acid sequence according to the invention or a vector comprising the abovementioned expression cassette, and the recombinant NCR which can be obtained from the transgenic organism by means of expression, are subject matter of the present invention. The use of transgenic organisms comprising an expression cassette according to the invention, for example for providing recombinant protein, and/or the use of these organisms in in-vivo assay systems are likewise subject matter of the present invention.

Preferred organisms for the recombinant expression are not only bacteria, yeasts, mosses, algae and fungi, but also eukaryotic cell lines.

Preferred mosses are Physcomitrella patens or other mosses described in Kryptogamen [cryptogams], Vol. 2, Moose, Farne [mosses, ferns], 1991, Springer Verlag (ISBN 3540536515).

Preferred within the bacteria are, for example, bacteria from the genus Escherichia, Erwinia, Flavobacterium, Alcaligenes or cyanobacteria, for example from the genus Synechocystis or Anabena.

Preferred yeasts are Candida, Saccharomyces, Schizosaccharomyces, Hansenula or Pichia.

Preferred fungi are Aspergillus, Trichoderma, Ashbya, Neurospora, Fusarium, Beauveria, Mortierella, Saprolegnia, Pythium, or other fungi described in Indian Chem Engr. Section B. Vol 37, No 1,2 (1995).

Preferred plants are selected in particular among monocotyledonous crop plants such as, for example, cereal species such as wheat, barley, sorghum or millet, rye, triticale, maize, rice or oats, and sugar cane. The transgenic plants according to the invention are, furthermore, in particular selected from among dicotyledonous crop plants such as, for example, Brassicaceae such as oilseed rape, cress, Arabidopsis, cabbages or canola; Leguminosae such as soya, alfalfa, pea, beans or peanut, Solanaceae such as potato, tobacco, tomato, eggplant or capsicum; steraceae such as sunflower, Tagetes, lettuce or Calendula; Cucurbitaceae such as melon, pumpkin/squash or zucchini, or linseed, cotton, hemp, flax, red pepper, carrot, sugar beet, or various tree, nut and grapevine species.

In principle, transgenic animals such as, for example, C. elegans, are also suitable as host organisms.

Also preferred is the use of expression systems and vectors which are available to the public or commercially available.

Those which must be mentioned for use in E. coli bacteria are the typical advantageous commercially available fusion and expression vectors pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which contains glutathione S transferase (GST), maltose binding protein or protein A, the pTrc vectors (Amann et al., (1988) Gene 69:301-315), “pKK233-2” by CLONTECH, Palo Alto, Calif. and the “pET” and “pBAD” vector series from Stratagene, La Jolla.

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, pGAPZ derivatives, pPICZ derivatives, and the vectors of the “Pichia Expression Kit” (Invitrogen Corporation, San Diego, Calif.). Vectors for use in filamentous fungi are described in: van den Hondel, C. A. M. J. J. & Punt, P. J. (1991) “Gene transfer systems and vector development for filamentous fungi, in: Applied-Molecular Genetics of Fungi, J. F. Peberdy, et al., eds., p. 1-28, Cambridge University Press: Cambridge.

As an alternative, insect cell expression vectors may also be used advantageously, for example for expression in Sf9, Sf21 or Hi5 cells, which are infected via recombinant baculoviruses. Examples of these are the vectors of the pAc series (Smith et al. (1983) Mol. Cell Biol. 3:2156-2165) and the pVL series (Lucklow and Summers (1989) Virology 170:31-39). Others which may be mentioned are the baculovirus expression systems “MaxBac 2.0 Kit” and “Insect Select System” by Invitrogen, Carlsbad or “BacPAK Baculovirus Expression System” by CLONTECH, Palo Alto, Calif. Insect cells are particularly suitable for overexpressing eukaryotic proteins since they effect posttranslational modifications of the proteins which are not possible in bacteria and yeasts. The skilled worker is familiar with the handling of cultured insect cells and with their infection for expressing proteins, which can be carried out analogously to known methods (Luckow and Summers, Bio/Tech. 6, 1988, pp. 47-55; Glover and Hames (eds) in DNA Cloning 2, A practical Approach, Expression Systems, Second Edition, Oxford University Press, 1995, 205-244).

Plant cells or algal cells are others which can be used advantageously for expressing genes. Examples of plant expression vectors can be found as mentioned above in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Moreover, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used in this context are of viral origin such as, for example, promoters of polyoma virus, adenovirus 2, cytomegalovirus or simian virus 40. Further prokaryotic and eukaryotic expression systems are mentioned in Chapter 16 and 17 in Sambrook et al., Molecular Cloning: A Laboratory Manual. 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The organisms transformed with an expression cassette according to the invention come under the term “transgenic organism according to the invention”.

The present invention furthermore relates to the use of NCR in a method for identifying herbicidally active compounds.

The method according to the invention for identifying herbicidally active compounds preferably comprises the following steps:
i. bringing an NCR into contact with one or more test compounds under conditions which permit binding of the test compound(s) to the NCR; and
ii. detecting whether the test compound binds to the NCR of i); or
iii. detecting whether the test compound reduces or blocks the activity of the NCR of i); or
iv. detecting whether the test compound reduces or blocks the transcription, translation or expression of the NCR of i).

The detection in accordance with step (ii) of the above method can be effected using techniques which identify the interaction between protein and ligand. In this context, either the test compound or the enzyme can contain a detectable label such as, for example, a fluorescent label, a radioisotope, a chemiluminescent label or an enzyme label. Examples of enzyme labels are horseradish peroxidase, alkaline phosphatase or luciferase. The subsequent detection depends on the label and is known to the skilled worker.

In this context, five preferred embodiments which are also suitable for high-throughput screening methods (HTS) in connection with the present invention, must be mentioned in particular:
1. The average diffusion rate of a fluorescent molecule as a function of the mass can be determined in a small sample volume via fluorescence correlation spectroscopy (FCS) (Proc. Natl. Acad. Sci. USA (1994) 11753-11575). FCS can be employed for determining protein/ligand interactions by measuring the changes in the mass, or the changed diffusion rate which this entails, of a test compound when binding to NCR. A method according to the invention can be designed directly for measuring the binding of a test compound labeled with a fluorescent molecule. As an alternative, the method according to the invention can be designed in such a way that a chemical reference compound which is labeled with a fluorescent molecule is displaced by further test compounds (“displacement assay”). The compounds which are identified in this manner may be suitable as inhibitors.
2. Fluoresence polarization exploits the characteristic of a quiescent fluorophore excited with polarized light to likewise emit polarized light. If, however, the fluorophore is allowed to rotate during the excited state, the polarization of the fluorescent light which is emitted is more or less lost. Under otherwise identical conditions (for example temperature, viscosity, solvent), the rotation is a function of molecule size, whereby findings regarding the size of the fluorophore-bound residue can be obtained via the reading (Methods in Enzymology 246 (1995), pp. 283-300). A method according to the invention can be designed directly for measuring the binding of a fluorescently labeled test compound to the NCR. As an alternative, the method according to the invention may also take the form of the “displacement assay” described under 1. The compounds identified in this manner may be suitable as inhibitors.
3. Fluorescent resonance energy transfer (FRET) is based on the irradiation-free energy transfer between two spatially adjacent fluorescent molecules under suitable conditions. A prerequisite is that the emission spectrum of the donor molecule overlaps with the excitation spectrum of the acceptor molecule. By labelling NCR with a fluorescent label and the binding test compound, the binding can be measured by means of FRET (Cytometry 34, 1998, pp. 159-179). As an alternative, the method according to the invention may also take the form of the “displacement assay” described under 1. An especially suitable embodiment of FRET technology is “Homogeneous Time Resolved Fluorescence” (HTRF) as can be obtained from Packard BioScience. The compounds which are identified in this manner may be suitable as inhibitors.
4. Surface-enhanced laser desorption/ionization (SELDI) in combination with a time-of-flight mass spectrometer (MALDI-TOF) makes possible the rapid analysis of molecules on a support and can be used for analyzing protein/ligand interactions (Worral et al., (1998) Anal. Biochem. 70:750-756). In a preferred embodiment, NCR is immobilized on a suitable support and incubated with the test compound. After one or more suitable wash steps, the test compound molecules which are additionally bound to NCR can be detected by means of the above-mentioned methodology and inhibitors can thus be selected. The compounds which are identified in this manner may be suitable as inhibitors.
5. The measurement of surface plasmon resonance is based on the change in the refractive index at a surface when a test compound binds to a protein which is immobilized to said surface. Since the change in the refractive index is identical for virtually all proteins and polypeptides for a defined change in the mass concentration at the surface, this method can be applied to any protein in principle (Lindberg et al. Sensor Actuators 4 (1983) 299-304; Malmquist Nature 361 (1993) 186-187). The measurement can be carried out for example with the automatic analyzer based on surface plasmon resonance which is available from Biacore (Freiburg) at a throughput of, currently, up to 384 samples per day. A method according to the invention can be designed directly for measuring the binding of a test compound to NCR. As an alternative, the method according to the invention may also take the form of the “displacement assay” described under 1. The compounds identified in this manner may be suitable as inhibitors.

All of the substances identified via the abovementioned methods can subsequently be checked for their herbicidal action in another embodiment of the method according to the invention.

Furthermore, there exists the possibility of detecting further candidates for herbicidal active ingredients by molecular modeling via elucidation of the three-dimensional structure of NCR by x-ray structure analysis. The preparation of protein crystals required for x-ray structure analysis, and the relevant measurements and subsequent evaluations of these measurements, the detection of a binding site in the protein, and the prediction of potential inhibitor structures are known to the skilled worker. In principle, an optimization of the compounds identified by the abovementioned methods is also possible via molecular modeling.

A preferred embodiment of the method according to the invention, which is based on steps i) and ii), consists in
i. expressing an NCR in a transgenic organism according to the invention, or growing an organism which naturally contains an NCR;
ii. bringing the NCR of step i) in the cell digest of the transgenic or nontransgenic organism, in partially purified form or in homogeneously purified form, into contact with a test compound; and
iii. selecting a compound which reduces or blocks the NCR activity, the activity of the NCR incubated with the test compound being compared with the activity of an NCR not incubated with a test compound.

The solution containing the NCR can consist of the lysate of the original organism or of the transgenic organism which has been transformed with an expression cassette according to the invention. If appropriate, the NCR can be purified partially or fully via customary methods. A general overview over current protein purification techniques is described, for example, in Ausubel, F. M. et al., Current Protocols in Molecular Biology, Greene Publishing Assoc. and Wiley-Interscience (1994); ISBN 0-87969-309-6. If NCR is obtained recombinantly, the protein which takes the form of a fusion with an affinity tag can be purified via affinity chromatography as is known to the skilled worker. NCR from human tissue may be purified, for example, by the method of Jollie et al. (Plant Physiol. 85, pp 457-462, 1987).

The NCR which is required for in-vitro methods can thus be isolated either by means of heterologous expression from a transgenic organism according to the invention or from an organism containing NCR activity, preferably from an undesired plant, the term “undesired plant” being understood as meaning the species mentioned at the outset.

To identify herbicidal compounds, NCR is incubated with a test compound. After a reaction time, the activity of an NCR incubated with the test compound is determined with the activity of an NCR not incubated with the test compound. If the NCR is inhibited, a significant decrease in activity in comparison with the activity of the noninhibited polypeptide according to the invention is observed, the result being a reduction of at least 10%, advantageously at least 20%, preferably at least 30%, especially preferably by at least 50%, up to 100% reduction (blocking). Preferred is an inhibition of at least 50% at test compound concentrations of 10⁻⁴ M, preferably at 10⁻⁵ M, especially preferably of 10⁻⁶ M, based on enzyme concentration in the micromolar range.

The enzyme activity of NCR can be determined for example by means of an activity assay in which the increase in the product, the decrease in the substrate (or starting material) or the decrease in a specific cofactor are determined as a function of a defined period of time, or by a combination of at least two of the abovementioned parameters.

Examples of suitable substrates are, for example, iron(III) cytochrome b5, potassium iron(III) cyanide, 2,6-dichlorophenolindophenol, methemerythrin, p-benzoquinone or 5-hydroxy-1,4-naphthoquinone, preferably iron(III) cytochrome b5, potassium iron(III) cyanide, 2,6-dichlorophenolindophenol, especially preferably iron(III) cytochrome b5, potassium iron(III) cyanide, very especially preferably potassium iron(III) cyanide and, for suitable cofactors, NADH. If appropriate, derivatives of the abovementioned compounds which contain a detectable label, such as, for example, a fluorescent label, a radioisotope or a chemiluminescent label, may also be used.

The amounts of substrate to be employed in the activity assay range from 0.5-10 mM, and the amounts of NADH range from 0.1-5 mM, based on 1-100 μg/ml enzyme.

In an especially preferred embodiment, the conversion of a substrate is monitored photometrically, using a modification of a method described by Mihara and Sato (Methods Enzymol., 52, 1978, pp. 102-108) which is based on the reduction of potassium iron(III) cyanide and photometric measurement at 420 nm.

A preferred embodiment of the method according to the invention which is based on steps i) and iii) consists of the following steps:
i. generation of a transgenic organism according to the invention;
ii. applying a test compound to the transgenic organism of i) and to a nontransgenic organism of the same genotype;
iii. determining the growth or the viability of the transgenic and the nontransgenic organisms after application of the test compound; and
iv. selection of test compounds which bring about a reduced growth or a reduced viability of the nontransgenic organism in comparison with the growth of the transgenic organism.

In this method, the polypeptide with the biological activity of an NCR is overexpressed in the transgenic organism of i). The transgenic organism thus shows an elevated NCR activity in comparison with a nontransgenic organism, elevated NCR activity of the transgenic organism being understood as meaning an activity which exceeds the activity of the nontransgenic organism of the same genus by at least 10%, preferably by at least 25%, especially preferably by at least 40%, very especially preferably by at least 50%.

In this context, the difference in growth in step iv) for the selection of a herbicidally active inhibitor amounts to at least 10%, preferably 20%, by preference 30%, especially preferably 40% and very especially preferably 50%.

The transgenic organism in this context is a bacterium, a yeast, a fungus, a plant or a eukaryotic cell line (derived from insects or from mammals such as, for example, mice), preferably plants, bacteria or yeasts, which can readily be transformed by means of customary techniques, such as Arabidopsis thaliana, Solanum tuberosum, Nicotiana tabacum or Saccharomyces cerevisiae, into which the sequence encoding a polypeptide according to the invention has been incorporated by transformation. These transgenic organisms thus show increased tolerance to compounds which inhibit the polypeptide according to the invention. Saccharomyces cerevisiae is in particular the organism of choice since its genome has been sequenced in its entirety and it can readily be used for the generation of “knock-out” mutants (for example Methods in Yeast Genetics, Kaiser, Michaelis, Mitchell (eds.) CSHL Press, Cold Spring Harbor Laboratory Press, 1994: 73-85) and the analogous NCR gene which is present in this organism can be silenced in a targeted manner.

However, the abovementioned method can also be used for identifying compounds with a growth-regulatory action. In this context, the transgenic organism employed is a plant. The method for identifying growth-regulatory compounds thus encompasses the following steps:
i. generation of a transgenic plant comprising a nucleic acid sequence according to the invention encoding an NCR;
ii. applying a test substance to the transgenic plant of i) and to a nontransgenic plant of the same variety;
iii. determining the growth or the viability of the transgenic and the nontransgenic plants after application of the test substance; and
iv. selection of test substances which bring about modified growth of the nontransgenic plant in comparison with the growth of the transgenic plant.

Step iv) involves the selection of test compounds which bring about a modified growth of the nontransgenic organism in comparison with the growth of the transgenic organism. Modified growth is understood as meaning, in this context, inhibition of the vegetative growth of the plants, which can manifest itself in particular in reduced longitudinal growth. Accordingly, the treated plants show stunted growth; moreover, their leaves are darker. In addition, modified growth is also understood as meaning a change of the course of maturation over time, the inhibition or promotion of lateral branched growth of the plants, shortened or extended developmental stages, increased standing ability, the growth of larger amounts of buds, flowers, leaves, fruits, seed kernels, roots and tubers, an increased sugar content in plants such as sugarbeet, sugar cane and citrus fruit, an increased protein content in plants such as cereals or soya, or stimulation of the latex flow in rubber trees. The skilled worker is familiar with the detection of such modified growth.

It is also possible, in the method according to the invention, to employ a plurality of test compounds in a method according to the invention. If a group of test compounds affects the target, then it is either possible directly to isolate the individual test compounds or to divide the group of test compounds into a variety of subgroups, for example when it consists of a multiplicity of different components, in order to reduce the number of the different test compounds in the method according to the invention. The method according to the invention is then repeated with the individual test compound or the relevant subgroup of test compounds. Depending on the complexity of the sample, the above-described steps can be carried out repeatedly, preferably until the subgroup identified in accordance with the method according to the invention only comprises a small number of test compounds, or indeed just one test compound.

All of the above-described methods for identifying herbicidally active inhibitors are hereinbelow referred to as “methods according to the invention”.

All of the compounds or substances which have been identified via the methods according to the invention can subsequently be tested in vivo for their herbicidal action. One possibility of testing the compounds for herbicidal action is to use duckweed, Lemna minor, in microtiter plates. Parameters which can be measured are modifications in the chlorophyll content and the photosynthesis rate. It is also possible to apply the compound directly to undesired plants, it being possible to identify the herbicidal action for example via restricted growth.

The method according to the invention can advantageously also be carried out in high-throughput methods, or high-throughput screening methods (HTS), which enable the simultaneous testing of a multiplicity of different compounds.

The use of supports which contain one or more of the nucleic acid molecules according to the invention, one or more of the vectors containing the nucleic acid sequence according to the invention, one or more transgenic organisms containing at least one of the nucleic acid sequences according to the invention or one or more (poly)peptides encoded by the nucleic acid sequences according to the invention lends itself to carrying out an HTS in practice. The support used can be solid or liquid, it is preferably solid and especially preferably a microtiter plate. The abovementioned supports are also subject matter of the present invention. In accordance with the most widely used technique, 96-well, 384-well and 1536-well microtiter plates which, as a rule, can comprise volumes of 200 μl, are used. Besides the microtiter plates, the further components of an HTS system which match the corresponding microtiter plates, such as a large number of instruments, materials, automatic pipetting devices, robots, automated plate readers and plate washers, are commercially available.

In addition to the HTS methods which are based on microtiter plates, what are known as free-format assays, or assay systems where there are no physical barriers between the samples, may also be used, as, for example, in Jayaickreme et al., Proc. Natl. Acad. Sci U.S.A. 19 (1994) 161418; Chelsky, “Strategies for Screening Combinatorial Libraries, First Annual Conference of The Society for Biomolecular Screening in Philadelphia, Pa. (Nov. 710, 1995); Salmon et al., Molecular Diversity 2 (1996), 5763 and U.S. Pat. No. 5,976,813.

The invention furthermore relates to herbicidally active compounds identified by the methods according to the invention. These compounds are hereinbelow referred to as “selected compounds”. They have a molecular weight of less than 1000 g/mol, advantageously less than 500 g/mol, preferably less than 400 g/mol, especially preferably less than 300 g/mol. Herbicidally active compounds have a Ki value of less than 1 mM, preferably less than 1 μM, especially preferably less than 0.1 μM, very especially preferably less than 0.01 μM.

Naturally, the selected compounds can also be present in the form of their agriculturally useful salts. Agriculturally useful salts which are suitable are mainly the salts of those cations, or the acid addition salts of those acids, whose cations, or anions, do not adversely affect the herbicidal action of the selected compounds.

If the selected compounds contain asymmetrically substituted α-carbon atoms, they may furthermore also be present in the form of racemates, enantiomer mixtures, pure enantiomers or, if they have chiral substituents, also in the form of diastereomer mixtures.

The selected compounds can be chemically synthesized substances or substances produced by microorganisms and can be found, for example, in cell extracts of, for example, plants, animals or microorganisms. The reaction mixture can be a cell-free extract or comprise a cell or cell culture. Suitable methods are known to the skilled worker and are described generally for example in Alberts, Molecular Biology the cell, 3^{rd} Edition (1994), for example Chapter 17. The selected compounds may also originate from extensive substance libraries.

Candidate test compounds can be expression libraries such as, for example, cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein).

The selected compounds can be used for controlling undesired vegetation, if appropriate also for the defoliation of, for example, potatoes or the desiccation of, for example, cotton, and as growth regulators. Herbicidal compositions comprising the selected compounds afford very good control of vegetation on noncrop areas. In crops such as wheat, rice, maize, soybean and cotton, they act against broad-leaved weeds and grass weeds without inflicting any significant damage on the crop plants. This effect is observed in particular at low application rates. The selected compounds can be used for controlling the harmful plants which have already been mentioned above.

Depending on the application method in question, selected compounds, or herbicidal compositions comprising them, can advantageously also be employed in a further number of crop plants for eliminating undesired plants. Examples of suitable crops are:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the selected compounds can also be used in crops which tolerate the action of herbicides owing to breeding, including recombinant methods. The generation of such crops is described hereinbelow.

The invention furthermore relates to a method of preparing the herbicidal composition which has already been mentioned above, which comprises formulating selected compounds with suitable auxiliaries to give crop protection products.

The selected compounds can be formulated for example in the form of directly sprayable aqueous solutions, powders, suspensions, also highly concentrated aqueous, oily or other suspensions or suspoemulsions or dispersions, emulsifiable concentrates, emulsions, oil dispersions, pastes, dusts, materials for spreading or granules by means of spraying, atomizing, dusting, spreading or pouring. The use forms depend on the intended use and the nature of the selected compounds; in any case, they should guarantee the finest possible distribution of the selected compounds. The herbicidal compositions comprise a herbicidally active amount of at least one selected compound and auxiliaries conventionally used in the formulation of herbicidal compositions.

For the preparation of emulsions, pastes or aqueous or oily formulations and dispersible concentrates (DC), the selected compounds can be dissolved or dispersed in an oil or solvent, it being possible to add further formulation auxiliaries for homogenization purposes. However, it is also possible to prepare liquid or solid concentrates from selected compound, if appropriate solvents or oil and, optionally, further auxiliaries, and these concentrates are suitable for dilution with water. The following can be mentioned: emulsifiable concentrates (EC, EW), suspensions (SC), soluble concentrates (SL), dispersible concentrates (DC), pastes, pills, wettable powders or granules, it being possible for the solid formulations to be either soluble or dispersible (wettable) in water. In addition, suitable powders or granules or tablets can additionally be provided with a solid coating which prevents abrasion or premature release of the active ingredient.

In principle, the term “auxiliaries” is understood as meaning the following classes of compounds: antifoam agents, thickeners, wetters, stickers, dispersants, emulsifiers, bactericides and/or thixotropic agents. The skilled worker is familiar with the meaning of the abovementioned agents.

SLs, EWs and ECs can be prepared by simply mixing the constituents in question; powders can be prepared by mixing or grinding in specific types of mills (for example hammer mills). DCs, SCs and SEs are usually prepared by wet milling, it being possible to prepare an SE from an SC by addition of an organic phase which may comprise further auxiliaries or selected compounds. The preparation is known. Powders, materials for spreading and dusts can advantageously be prepared by mixing or concomitantly grinding the active substances together with a solid carrier. Granules, for example coated granules, impregnated granules and homogeneous granules, can be prepared by binding the selected compounds to solid carriers. The skilled worker is familiar with further details regarding their preparation, which are mentioned for example in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

The skilled worker is familiar with a multiplicity of inert liquid and/or solid carriers which are suitable for the formulations according to the invention, such as, for example, liquid additives such as mineral oil fractions of medium to high boiling point such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol and cyclohexanol, ketones such as cyclohexanone, or strongly polar solvents, for example amines such as N-methylpyrrolidone or water.

Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The skilled worker is familiar with a multiplicity of surface-active substances (surfactants) which are suitable for the formulations according to the invention such as, for example, alkali metal salts, alkaline earth metal salts or ammonium salts of aromatic sulfonic acids, for example lignosulfonic acid, phenolsulfonic acid, naphthalenesulfonic acid and dibutylnaphthalenesulfonic acid, and of fatty acids, of alkyl- and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

The herbicidal compositions, or the selected compounds, can be applied pre- or post-emergence. If the selected compounds are less well tolerated by certain crop plants, application techniques may be used in which the selected compounds are sprayed, with the aid of the spraying apparatus, in such a way that they come into as little contact, if any, with the leaves of the sensitive crop plants while the selected compounds reach the leaves of undesired plants which grow underneath, or the bare soil surface (post-directed, lay-by).

Depending on the intended aim, the season, the target plants and the growth stage, the application rates of selected compounds amount to 0.001 to 3.0, preferably 0.01 to 1.0 kg/ha.

Providing the herbicidal target furthermore makes possible a method for identifying a protein with the biological activity of an NCR which is not inhibited, or inhibited to a limited extent only, by a herbicide which has NCR as its selective action, for example the herbicidally active selected compounds. A protein which differs in this way from NCR is hereinbelow referred to as NCR variant, which is encoded by a nucleic acid sequence which
i) encodes a polypeptide with the biological activity of an NADH-dependent cytochrome b5 reductase which is not inhibited by herbicidally active substances which inhibit NCR and which have been identified by the abovementioned methods; and
ii) which comprises a functional equivalent of the nucleic acid sequence SEQ ID NO:1 with at least 52% identity with SEQ ID NO:1; or which can be derived by backtranslating the amino acid sequence of a functional equivalent of SEQ ID NO:2 which has at least 39% identity with SEQ ID NO:2.

In a preferred embodiment, the abovementioned method for generating nucleic acid sequences encoding NCR variants of nucleic acid sequences comprise the following steps:
a) expression, in a heterologous system or in a cell-free system, of the proteins encoded by the abovementioned nucleic acids;
b) randomized or site-directed mutagenesis of the protein by modification of the nucleic acid;
c) measuring the interaction of the modified gene product with the herbicide;
d) identification of derivatives of the protein which show less interaction;
e) assaying the biological activity of the protein after application of the herbicide;
f) selection of the nucleic acid sequences which show a modified biological activity toward the herbicide.

The functional SEQ ID NO:1 equivalents according to the invention have at least 52%, 53%, 54%, 55%, 56%, 57%, 58%, 59%, preferably at least 60%, 61%, 62%, 63%, 64%, 65%, 66%, 67%, 68%, 69% and 70%, preferably at least 71%, 72%, 73%, 74%, 75%, 76%, especially preferably at least 77%, 78%, 79%, 80%, 81%, 82%, 83%, 84%, 85%, 86%, 87%, 88%, 89%, 90%, very especially preferably at least 91%, 92%, 93%, 94%, 95%, 96%, 97%, 98%, 99% homology with SEQ ID NO:1.

The functional SEQ ID NO:2 equivalents according to the invention have at least 39%, 40%, 41%, 42%, 43%, 44%, 45%, 46%, 47%, 48%, 49%, 50%, 51%, 52%, 53%, 54%, 55%, 56%, 57%, 58%, 59%, preferably at least 60%, 61%, 62%, 63%, 64%, 65%, 66%, 67%, 68%, 69% and 70%, preferably at least 71%, 72%, 73%, 74%, 75%, 76%, 77%, 78%, 79%, 80%, 81%, 82%, 83%, 84%, 85%, 86%, especially preferably at least 87%, 88%, 89%, 90%, 91%, 92%, 93%, very especially preferably at least 94%, 95%, 96%, 97%, 98%, 99% homology with SEQ ID NO:2.

The sequences which are selected by the above-described method are advantageously introduced into an organism. The invention therefore furthermore relates to an organism prepared by this method The organism is preferably a plant, especially preferably one of the above-defined crop plants.

Thereafter, intact plants are regenerated and the resistance to the selected compound is verified in intact plants.

Modified proteins and/or nucleic acids which are capable of conferring, in plants, resistance to the selected compounds can also be generated from the abovementioned nucleic acid sequences via site-directed mutagenesis; this mutagenesis allows for example the stability and/or activity of the target protein or the characteristics such as binding and action of the abovementioned inhibitors according to the invention to be improved or modified in a highly targeted manner.

A site-directed mutagenesis method in plants which can advantageously be used has been described for example by Zhu et al. (Nature Biotech., Vol. 18, May 2000: 555-558).

Moreover, modifications can be obtained via the PCR method described by Spee et al. (Nucleic Acids Research, Vol. 21, No. 3, 1993: 777-78) using dITP for random mutagenesis, or by the further improved method of Rellos et al. (Protein Expr. Purif., 5, 1994: 270-277).

A further possibility of generating these modified proteins and/or nucleic acids is an in-vitro recombination technique for olecular evolution, which has been described by Stemmer et al. (Proc. Natl. Acad. Sci. USA, Vol. 91, 1994; 10747-10751), or the combination of the PCR and recombination method, which has been described by Moore et al. (Nature Biotechnology Vol. 14, 1996: 458-467).

A further way of mutating proteins is described by Greener et al. in Methods in Molecular Biology (Vol. 57, 1996: 375-385). EP-A-0 909 821 describes a method of modifying proteins using the microorganism E. coli XL-1 Red. Upon replication, this microorganism generates mutations in the introduced nucleic acids and thus leads to a modification of the genetic information. Advantageous nucleic acids and the proteins encoded by them can be identified readily via isolating the modified nucleic acids or the modified proteins and carrying out resistance testing. After introduction into plants, they can manifest resistance therein and thus lead to resistance to the herbicides.

Further methods of mutagenesis and selection are, for example, methods such as the in vivo mutagenesis of seeds or pollen and selection of resistant alleles in the presence of the inhibitors according to the invention, followed by the genetic and molecular identification of the modified, resistant allele. Furthermore the mutagenesis and selection of resistances in cell culture by growing the culture in the presence of successively increasing concentrations of the inhibitors according to the invention. In doing so, the increase in the spontaneous mutation rate by chemical/physical mutagenic treatment may be exploited. As described above, modified genes may also be isolated using microorganisms which have an endogenous or recombinant activity of the proteins encoded by the nucleic acids used in the method according to the invention, which microorganisms are sensitive to the inhibitors identified in accordance with the invention. Growing the microorganisms on media with increasing concentrations of inhibitors according to the invention permits the selection and evolution of resistant variants of the targets according to the invention. The frequency of the mutations, in turn, can be increased by mutagenic treatments.

In addition, methods are available for the targeted modification of nucleic acids (Zhu et al. Proc. Natl. Acad. Sci. USA, Vol. 96, 8768-8773 and Beethem et al., Proc. Natl. Acad. Sci. USA, Vol 96, 8774-8778). These methods make it possible to replace, in the proteins, those amino acids which are of importance for binding inhibitors by functionally equivalent amino acids which, however, inhibit the binding of the inhibitor.

The invention therefore furthermore relates to a method of generating nucleic acid sequences which encode gene products with a modified biological activity, the biological activity being modified such that an increased activity is present. Increased activity is to be understood as meaning an activity which is increased over the original organism, or over the original gene product, by at least 10%, preferably by at least 30%, especially preferably by at least 50%, very especially preferably by at least 100%. Moreover, the biological activity may have been modified such that the substances and/or compositions according to the invention no longer, or no longer correctly, bind to the nucleic acid sequences and/or the gene products encoded by them. No longer, or no longer correctly, is to be understood as meaning for the purposes of the invention that the substances bind by at least 30% less, preferably by at least 50% less, especially preferably by at least 70% less, very especially preferably by at least 80% less or not at all to the modified nucleic acids and/or gene products in comparison with the original gene product or the original nucleic acids.

Yet another aspect of the invention therefore relates to a transgenic plant which has been transformed with a nucleic acid sequence which encodes a gene product with a modified biological activity, or with a nucleic acid sequence encoding an NCR variant. Transformation methods are known to the skilled worker and examples have been given further above.

Genetically modified transgenic plants which are resistant to the substances found by the methods according to the invention and/or to compositions comprising these substances can also be generated by transformation followed by overexpression of a nucleic acid sequence according to the invention. This is why the invention furthermore relates to a method for generating transgenic plants which are resistant to substances which have been found by a method according to the invention, which comprises overexpressing, in these plants, nucleic acids encoding an NCR variant. A similar method is described, for example, in Lermantova et al., Plant Physiol., 122, 2000: 75-83.

The above-described methods according to the invention for generating resistant plants make possible the development of novel herbicides which have as complete as possible an action which is independent of the plant species (what are known as nonselective herbicides), in combination with the development of useful plants which are resistant to the nonselective herbicide. Useful plants which are resistant to nonselective herbicides have already been described on several occasions. In this context, one can distinguish between several principles for achieving a resistance:
a) generation of resistance in a plant via mutation methods or recombinant methods by markedly overproducing the protein which acts as target for the herbicide and by the fact that, owing to the large excess of the protein which acts as target for the herbicide, the function exerted by this protein in the cell is even retained after application of the herbicide.
b) modification of the plant such that a modified version of the protein which acts as target of the herbicide is introduced and that the function of the newly introduced modified protein is not adversely affected by the herbicide.
c) modification of the plant such that a novel protein/a novel RNA is introduced wherein the chemical structure of the protein or of the nucleic acid, such as of the RNA or the DNA, which structure is responsible for the herbicidal action of the low-molecular-weight substance, is modified so that, owing to the modified structure, a herbicidal action can no longer unfold, that is to say the interaction of the herbicide with the target can no longer take place.
d) the function of the target is replaced by a novel gene which has been introduced into the plant, and what is known as an “alternative pathway” is created.
e) the function of the target is taken over by another gene, or its gene product, present in the plant.

The skilled worker is familiar with alternative methods for identifying the homologous nucleic acids, for example in other plants with similar sequences, such as, for example, using transposons. The present invention therefore also relates to the use of alternative insertion mutagenesis methods for the insertion of foreign nucleic acids into the nucleic acid sequences SEQ ID NO:1, SEQ ID NO:3 or SEQ ID NO:5 in sequences derived from these sequences on the basis of the genetic code and/or in their derivatives in other plants.

The transgenic plants are generated with one of the above-described embodiments of the expression cassette according to the invention by customary transformation methods which have likewise been described above.

The expression efficiency of the recombinantly expressed NCR can be determined for example in vitro by shoot meristem propagation or by a germination test. Moreover, the expression of the NCR gene whose nature and level has been modified, and its effect on the resistance to NCR inhibitors, can be tested on test plants in greenhouse experiments.

The invention is illustrated in greater detail by the examples which follow, which are not to be considered as limiting.

General DNA Manipulation and Cloning Methods

Cloning methods such as, for example, restriction cleavages, agarose gel electrophoresis, purification of DNA fragments, transfer of nucleic acids to nitrocellulose and nylon membranes, linking DNA fragments, transformation of Escherichia coli cells, bacterial cultures and sequence analysis of recombinant DNA were carried out as described by Sambrook et al. (1989) (Cold Spring Harbor Laboratory Press: ISBN 0-87969-309-6) and Ausubel, F. M. et al., Current Protocols in Molecular Biology, Greene Publishing Assoc. and Wiley-Interscience (1994); ISBN 0-87969-309-6.

Molecular-biological standard methods for plants and plant transformation methods are described in Schultz et al., Plant Molecular Biology Manual, Kluwer Academic Publishers (1998), Reither et al., Methods in Arabidopsis Research, World scientific press (1992) and Arabidopsis: A Laboratory Manual (2001), ISBN 0-87969-573-0.

The bacterial strains used hereinbelow (E. coli DH5α, XL-1 blue, XL10 Gold, BL21DE(3), JM 109) were obtained from Stratagene, BRL Gibco or Invitrogen, Carlsberg, Calif. The vectors used for cloning were pCR T7CT TOPO, pCR T7/NT TOPO and pCR 2.1 TOPO from Invitrogen and pUC 19 from Amersham Pharmacia (Freiburg), pBinAR (Höfgen and Willmitzer, Plant Science 66, 1990, 221-230) and pMALc2x (New England Biolabs).

### EXAMPLE 1

### Generation of an Arabidopsis Plant Transformation Vector

The primer pair Rei 156/Rei 157

(SEQ ID NO:5)

Rei 156: 5′-TATACCCGGGATGGATACCGAGTTTCTCCGAA-3′
and

(SEQ ID NO:6)

Rei 157: 5′-TATACCCGGGGAACTGGAATTGCATCTCCGGA-3′

was derived from a cDNA sequence encoding Arabidopsis thaliana NCR (Accession Number AB007799; Mizutani, M. and Fukuchi-Mizutani, M., 1997). Thereafter, the primers Rei 156/157 were used in a PCR reaction for amplifying a cDNA fragment from an Arabidopsis thaliana cDNA library (Stratagene). The PCR was carried out under the conditions stated in Table 1.

TABLE 1

| Temperature [° C.] | Time [sec] | Number or cycles |
|---|---|---|
| 95 | 300 | 1 |
| 95 | 60 | 34 |
| 58 | 90 | |
| 72 | 300 | |
| 72 | 600 | 1 |

Following purification via agarose gel electrophoresis, the resulting fragment (SEQ ID NO:1) was cloned into the vector pPCRScript (pPCRScript-NCR), following the manufacture's instructions. Sequencing allowed the verification of the identity of the full-length Arabidopsis NCR cDNA clone.

The vector pBinAR (Höfgen and Willmitzer 1990, Plant Science 66, 221-230) was cleaved using SmaI and ligated to the NCR fragment isolated from the vector pPCRScript-NCR, which had been isolated via SmaI (pBinAR-NCR). The ligation was transformed into XL10 Gold E. coli cells and pBinAR-NCR-containing clones were identified using a digoxigenin-labeled NCR probe (Roche) with the aid of anti-digoxigenin antibodies. The antisense orientation of the NCR-cDNA in pBinAR-NCR was confirmed by sequencing and using PCR reactions with 2 different primer pairs (Rei 143 and Rei 196, and Rei 144 and Rei 195, respectively) under the conditions stated in Table 2.

TABLE 2

| Temperature [° C.] | Time [sec] | Number of cycles |
|---|---|---|
| 95 | 120 | 1 |
| 95 | 60 | 34 |
| 58 | 60 | |
| 72 | 120 | |
| 72 | 300 | 1 |

The antisense orientation of NCR was confirmed using the primers

| | |
|---|---|
| Rei 143: 5′-GCTATGACCATGATTACGCC-3′ | (SEQ ID NO:7) |
| and | |
| Rei 196: 5′-TGAGACATCCGTCCTTGC-3′ | (SEQ ID NO:8) |

via the appearance of a 740 bp DNA fragment and with the primers

| | |
|---|---|
| Rei 144: 5′-ACGTTGTAAAACGACGGCCA-3′ | (SEQ ID NO:9) |
| and | |
| Rei 195: 5′-CCGACTACGTTAGACTCTG-3′ | (SEQ ID NO:10) |

via the appearance of an 885 bp DNA fragment.

### EXAMPLE 2

### Transformation and Analysis of Arabidopsis Plants

The construct pBinAR-NCR was transformed into the agrobacterial strain pGV 2260. A positively transformed agrobacterial colony was employed for transforming Arabidopsis plants. The detection of the presence of the construct pBinAR-NCR in an agrobacterial colony was via PCR using the primers Rei 156 and Rei 157 via PCR under the conditions stated in Table 2. The DNA template used was an amount of an agrobacterial colony which was taken directly from the agar plate.

A 4 ml culture on LB medium (LB medium: 10 g/l peptone, 5 g/l yeast extract, 10 g/l NaCl; pH 7.0; 80 mg/l kanamycin/l and 25 mg/l rifampicin) was inoculated by a single colony of positively transformed agrobacteria on a plate and the culture was incubated overnight at 28° C. Thereafter, a 400 ml culture in LB medium (LB medium supplemented with 80 mg kanamycin/ml and 25 mg/ml rifampicin) was inoculated with this culture. After incubation for 12 hours at 28° C. and 220 rpm, the culture was precipitated (8.000 rpm, 20 min.) and resuspended in transformation medium (½ MS medium as described by Murashige T. and Skoog F. 1962. Physiologia Plantarum. 15: 473-497; Owen H. R. and Miller A. R. 1992. Plant Cell, Tissue and Organ Culture 28: 147-150; 0.5 g/l 2-(N-morpholino)ethanesulfonic acid), pH 5.8; 50 g/l sucrose). Flowering Arabidopsis plants were immersed in the resulting suspension approximately 5 times on three occasions at 2.5 day intervals and subsequently transferred to pots containing moist soil. After incubation for 6 weeks under long-day conditions in controlled-environment cabinets (daytime temperature 22-24° C., night time temperature 19° C.; relative atmospheric humidity 65%), the seeds of the plant were harvested.

### EXAMPLE 3

### Analysis of the Transgenic Plants

To carry out the analysis, seeds of the transformed plants of Example 2 were on agar selection plates (2.15 g/l Murashige+Skoog micro and macro elements (Fa. DUCHEFA; as described by Murashige T. and Skoog F. 1962. Physiologia Plantarum. 15: 473-497; Owen H. R.; Miller A. R. 1992. Plant Cell, Tissue and Organ Culture 28: 147-150) 0.1 g/l myo-inositol, 0.5 g/l MES, 10 g/l sucrose, pH 5.7, 1 ml % by weight vitamin B5, 50 μg/l kanamycin; 15 g/l agar agar).

Plants which have grown on the selection plates were transferred into soil after 3-4 weeks and incubated for 4-8 weeks in controlled-environment cabinets under long-day conditions (daytime temperature 22-24° C., night time temperature 19° C.; relative atmospheric humidity 65%). After 6 weeks, the seeds were harvested. The integration of the antisense NCR gene into the genome of the transgenic plants was verified by PCR under the conditions stated in Table 3.

TABLE 3

| Temperature [° C.] | Time [sec] | Number of cycles |
|---|---|---|
| 95 | 120 | 1 |
| 95 | 60 | 34 |
| 45-50 | 60 | |
| 72 | 120 | |
| 72 | 300 | 1 |

The template used was genomic DNA (isolation by means of the “DNeasy Plant Mini” kits from QIAGEN, following the manufacturer's instructions) which had been isolated from leaf material of the transgenic lines in question. The NCR antisense pBinAR construct used for the transformation was used as positive control. By deliberately choosing the primers, both the intrinsic genomic gene, which contains an intron and is thus longer than the antisense NCR cDNA and the antisense NCR cDNA itself were detected. In the presence of the antisense NCR cDNA in the genome of the transgenic plants, the expected fragment lengths for the genomic NCR (with an intron) amounted to approx. 1800 bp when using the primers

(SEQ ID NO:11)

Rei 524: 5′-TTCGTTGCTTTCGTCGCCGTT-3′
and

(SEQ ID NO:12)

Rei 525: 5′-GTTTGCAGCCATGGCCTTGTT-3′

and 750 bp for the antisense NCR cDNA when using the primers

(SEQ ID NO:11)

Rei 524: 5′-TTCGTTGCTTTCGTCGCCGTT-3′
and

(SEQ ID NO:13)

Rei 527: 5′-GGCGGGAAACGACAATCTGATC-3′.

Transgenic plants which contained the construct pBinAR-NCR in antisense orientation showed substantial accumulation of anthocyanin, that is to say severely stressed leaves and veins, chlorotic leaves and drastically reduced growth. Thus, the fresh weight of transgenic plants (TO generation) only amounted to 1-10% of the fresh weight of wild-type plants after 6 weeks cultivation in the soil under long-day conditions in controlled-environment cabinets (daytime temperature 22-24° C., night time temperature 19° C.; relative atmospheric humidity 65%). The seeds which developed in the pods of the plants of the transformed TO generation were shriveled and did not germinate in 100% of all cases.

Thus, it was, surprisingly, demonstrated for the first time that the natural expression of NCR encoding sequences is essential for plants and that reduced expression leads to damage as stated for the abovementioned phenotypes. Thus, it was demonstrated that NCR is suitable as a target for herbicides.

### EXAMPLE 4

### Expression in E. coli

To generate active protein with plant NCR activity, an Arabidopsis cDNA encoding an NCR (Genbank Accession Number: AB007799) was overexpressed in E. coli bacteria. To this end, the NCR-encoding nucleic acid sequence was amplified via PCR (for example as described by Sambrook, J. et al. (1989) “Molecular cloning: A laboratory manual”, Cold Spring Harbor Laboratory Press; 34 cycles; annealing temperature 60° C.; polymerization time 2 min) under standard conditions using pBinAR-NCR as template and the primers

(SEQ ID NO:14)

Rei 153: 5′- TATAGAATTCATGGATACCGAGTTTCTCCGAA-3′
(EcoRI)

(SEQ ID NO:15)

Rei 483: 5′- TATACTGCAGTCAGAACTGGAATTGCATCTCCGG-3′
(PstI)

comprising the restriction enzyme cleavage sites EcoRI and PstI and cloned into the vector pMAL-c2x (New England Biolabs) via the restriction enzyme cleavage sites EcoRI and PstI (pMAL-c2x-NCR).

The pMAL-c2x-NCR constructs were transformed into the E. coli strain JM109 (Stratagene) and NCR was expressed following the manufacturer's instructions via IPTG in the form of a fusion protein with maltose binding protein (NCR-MBP). The protein was purified via affinity chromatography using a maltose column as described by the manufacturer New England Biolabs.

### EXAMPLE 5

### In Vitro Assay Systems

The NCR activity was determined by the method of Mihara and Sato (Methods Enzymol., 52, 1978, pp. 102-108) with NCR which had been expressed recombinantly as described in Example 4 (Fukuchi-Mizutani et al., Plant Physiol. 119, pp. 353-361, 1999) or by the method described by Jollie et al. (Plant Physiol. 85, pp. 457-462, 1987).

When following the method of Mihara and Sato (Methods Enzymol., 52, 1978, pp. 102-108), 1-10 μg of purified NCR-MBP protein are treated with 1 mM potassium iron(III) cyanide in 100 μl of buffer (100 mM K₂HPO—/KH₂PO₄ buffer (mixture of equal parts). The reaction is started by addition of 0.3 mM β-NADH.

The reduction of potassium iron(III) cyanide is measured photometrically at 420 nm and 25° C. over a period of 5 to 15 minutes.

### EXAMPLE 6

### Identification of a Functional Analog from Tobacco

To generate a cDNA library (hereinbelow referred to as binary cDNA library”) in a vector which can be used directly for the transformation of plants, mRNA was isolated from various plant tissues and transcribed into double-stranded cDNA using the TimeSaver cDNA synthesis kit (Amersham Pharmacia Biotech, Freiburg). The cDNA first-strand synthesis was carried out using T₁₂₋₁₈ oligonucleotides, following the manufacturer's instructions. After size fractionation and ligation of EcoRI-NotI adapters following the manufacturer's instructions and filling up the overhangs with Pfu DNA Polymerase (Stratagene), the cDNA population was normalized. This was done following the method of Kohci et al., 1995, Plant Journal 8, 771-776, the cDNA being amplified by PCR with the oligonucleotide N1 under the conditions stated in Table 4.

TABLE 4

| Temperature [° C.] | Time [sec] | Number of cycles |
|---|---|---|
| 94 | 300 | 1 |
| 94 | 8 | 10 |
| 52 | 60 | |
| 72 | 180 | |
| 94 | 8 | 10 |
| 50 | 60 | |
| 72 | 180 | |
| 94 | 8 | 10 |
| 48 | 60 | |
| 72 | 180 | |
| 72 | 420 | 1 |

The resulting PCR product was bound to the column matrix of the PCR Purification Kit (Qiagen, Hilden) and eluted with 300 mM NaP buffer, pH 7.0, 0.5 mM EDTA, 0.04% SDS. The DNA was denatured for 5 minutes in a boiling water bath and subsequently renatured for 24 hours at 60° C. 50 μl of the DNA were applied to a hydroxyapatite column and the column was washed 3 times with 1 ml of 10 mM NaP buffer, pH 6.8. The bound single-stranded DNA was eluted with 130 mM NaP buffer, pH 6.8, precipitated with ethanol and dissolved in 40 μl of water. 20 μl of this was used for a further PCR amplification as described above. After further ssDNA concentration, a third PCR amplification was carried out as described above.

The plant transformation vector for taking up the cDNA population which had been generated as described above was generated via restriction enzyme cleavage of the vector pUC18 with SbfI and BamHI, purification of the vector fragment followed by filling up of the overhangs with Pfu DNA polymerase and religation with T4 DNA ligase (Stratagene). The resulting construct is hereinbelow termed pUC18SbfI-.

The vector pBinAR was first cleaved with NotI, the ends were filled up and the vector was religated, cleaved with SbfI, the ends were filled up and the vector was religated and subsequently cleaved with EcoRI and HindIII. The resulting fragment was ligated into a derivative of the binary plant transformation vector pPZP (Hajdukiewicz, P, Svab, Z, Maliga, P., (1994) Plant Mol Biol 25:989-994) which enables the transformation of plants by means of agrobacterium and mediates kanamycin resistance in transgenic plants. The construct generated thus is hereinbelow termed pSun12/35S.

pUC18SbfI- was used as template in a polymerase chain reaction (PCR) with the oligonucleotides V1 and V2 (see Table 3) and Pfu DNA polymerase. The resulting fragment was ligated into the SmaI-cut pSun12/35S, giving rise to pSunblues2. Following cleavage with NotI, dephosphorylation with shrimp alkaline phosphatase (Roche Diagnostics, Mannheim) and purification of the vector fragment, pSunblues2 was ligated with the normalized, likewise NotI-cut cDNA population. Following transformation into

E. coli Xl-1blue (Stratagene), the resulting clones were deposited into microtiter plates. The binary cDNA library contains cDNAs in “sense” and in “antisense” orientation under the control of the cauliflower mosaic virus 35S promoter, and, after transformation into tobacco plants, these cDNAs can, accordingly, lead to “cosuppression” and “antisense” effects.

TABLE 3
Oligonucleotides used

| | |
|---|---|
| Oligonucleotide | Nucleic acid sequence |

| | |
|---|---|
| N1 | 5′-AGAATTCGCGGCCGCT-3′ |
| (SEQ ID NO:16) | |
| V1 (PWL93not) | 5′-CTCATGCGGCCGCGCGCAACGCAATTAAT |
| (SEQ ID NO:17) | GTG-3′ |
| V2 (pWL92) | 5′-TCATGCGGCCGCGAGATCCAGTTCGATGT |
| (SEQ ID NO:18) | AAC-3′ |
| G1 (35S) | 5′-GTGGATTGATGTGATATCTCC-3′ |
| (SEQ ID NO:19) | |
| G2 (OCS) | 5′-GTAAGGATCTGAGCTACACAT-3′ |
| (SEQ ID NO:20) | |

An NCR-encoding sequence was identified via a digoxygenin-labeled probe generated using the DIG DNA Labeling Mix (Roche, Mannheim) following the manufacturer's instructions, the plasmid pMAL-c2x-NCR being amplified under standard conditions by PCR with the primers

(SEQ ID NO:21)

Rei 111: 5′-ATGGATACCGAGTTTCTCCGAA-3′
and

(SEQ ID NO:22)

Rei 222: 5′-AACTGGAATTGCATCTCCGGA-3′.

The probe generated thus was used for screening the Nicotiana tabacum cDNA library. The cDNA library was plated using a titer of 2.5×10⁵ plaque-forming units and analyzed with the aid of the plaque screening method (T. Maniatis, E. F. Fritsch and J. Sambrook, Molecular Cloning: A Laboratory Manual, Cold Spring Harbor Laboratory, Cold Spring Harbor, N.Y. 1989). 12 phage populations were isolated and used in a second screening step, whereby genetically uniform phage populations were isolated which were used for the in-vivo excision. Restriction analysis revealed no differences between the cDNA clones, and four clones with the largest insertions were selected for sequencing. The sequence data of these clones gave SEQ ID NO:3, which has 77% identity with SEQ ID NO:1.

We claim:

1. The use of a polypeptide with the biological activity of an NADH-dependent cytochrome b5 reductase encoded by a nucleic acid sequence consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation; or
d) a functional equivalent of the nucleic acid sequence SEQ ID NO:1 with at least 52% identity with SEQ ID NO:1,
as target for identifying herbicidally active compounds.

2. A plant nucleic acid sequence encoding a polypeptide with the biological activity of an NADH-dependent cytochrome b5 reductase comprising a part-region encompassing:
a) a nucleic acid sequence with the nucleic acid sequence shown in SEQ ID NO:3; or
b) a nucleic acid sequence which, on the basis of the degeneracy of the genetic code, can be deduced from the amino acid sequence shown in SEQ ID NO:4 by back translation; or
c) a functional equivalent of the nucleic acid sequence SEQ ID NO:3 with at least 77% identity with SEQ ID NO:3;
d) a nucleic acid sequence which, on the basis of the degeneracy of the genetic code, can be deduced from the amino acid sequence of a functional equivalent of SEQ ID NO:4, which has at least 87% identity with SEQ ID NO:4, by back translation.

3. A polypeptide with the biological activity of an NADH-dependent cytochrome b5 reductase as target for herbicides, encoded by a nucleic acid molecule as claimed in claim 2.

4. A method for detecting functional analogues of SEQ ID NO:1
a) by preparing a probe and subsequently screening a genomic library or a cDNA library of the species in question; or
b) by a computer search for analogous sequences in electronic databases.

5. An expression cassette comprising
a) genetic control sequences in operable linkage with a nucleic acid sequence as claimed in claim 2; or
b) additional functional elements; or
c) a combination of a) and b).

6. A vector comprising an expression cassette as claimed in claim 5.

7. A nonhuman transgenic organism comprising at least one nucleic acid sequence encoding a polypeptide with the biological activity of an NADH-dependent cytochrome b5 reductase as claimed in claim 2, an expression cassette as claimed in claim 5 or a vector as claimed in claim 6 selected from among bacteria, yeasts, fungi, animal cells or plant cells.

8. The use of a polypeptide with the biological activity of an NADH-dependent cytochrome b5 reductase encoded by a nucleic acid sequence consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a functional equivalent of the nucleic acid sequence SEQ ID NO:1 with at least 52% identity with SEQ ID NO:1;
d) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation,
in a method for identifying herbicidally active compounds.

9. A method for identifying herbicidally active compounds, encompassing the following steps:
i. bringing a polypeptide with the biological activity of an NADH-dependent cytochrome b5 reductase encoded by a nucleic acid sequence consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a functional equivalent of the nucleic acid sequence SEQ ID NO:1 with at least 52% identity with SEQ ID NO:1; or
d) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation
into contact with one or more test compounds under conditions which allow the test compound(s) to bind to the NADH-dependent cytochrome b5 reductase; and
ii. detecting whether the test compound binds to the NADH-dependent cytochrome b5 reductase of i); or
iii. detecting whether the test compound reduces or blocks the activity of the NADH-dependent cytochrome b5 reductase of i); or
iv. detecting whether the test compound reduces or blocks the transcription, translation or expression of the NADH-dependent cytochrome b5 reductase of i).

10. A method as claimed in claim 9, which comprises
i. either expressing, in a nonhuman transgenic organism, NADH-dependent cytochrome b5 reductase which is encoded by a nucleic acid sequence consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a functional equivalent of the nucleic acid sequence SEQ ID NO:1 with at least 52% identity with SEQ ID NO:1; or
d) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation
or culturing an organism which naturally contains NADH-dependent cytochrome b5 reductase;
ii. bringing the NADH-dependent cytochrome b5 reductase of step i) in a cell digest of the transgenic or nontransgenic organism, in partially or homogeneously purified form, into contact with a test compound; and
iii. selecting a test compound which reduces or blocks the activity of the NADH-dependent cytochrome b5 reductase of step i), where the activity of the NADH-dependent cytochrome b5 reductase incubated with the test compound is compared with the activity of an NADH-dependent cytochrome b5 reductase which is not incubated with a test compound.

11. A method as claimed in claim 10, wherein, in step iii), the activity of the NADH-dependent cytochrome b5 reductase is determined by using iron(III) cytochrome b5, potassium iron(III)-cyanide, 2,6-dichlorophenolindophenol, methemerythrin, p-benzoquinone or 5-hydroxy-1,4-naphthoquinone as the substrate.

12. A method as claimed in claim 9, which encompasses the following steps:
i. generating a nonhuman transgenic organism as claimed in claim 7 or a transgenic organism comprising a nucleic acid sequence encoding a polypeptide with the biological activity of an NADH-dependent cytochrome b5 reductase consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a functional equivalent of the nucleic acid sequence SEQ ID NO:1 with at least 52% identity with SEQ ID NO:1; or
d) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation;
ii. applying a test substance to the transgenic organism of claim i) and to a nontransgenic organism of the same genotype;
iii. determining the growth or the viability of the transgenic and of the nontransgenic organisms after application of the test substance; and
iv. selection of test substances which bring about a reduced growth or a reduced viability of the nontransgenic organism in comparison with the growth of the transgenic organism.

13. A method as claimed in claim 12, which is carried out in a plant organism or a yeast.

14. A method for identifying growth-regulatory compounds, comprising the following steps:
i. generation of a transgenic plant comprising a nucleic acid sequence encoding a polypeptide with the biological activity of an NADH-dependent cytochrome b5 reductase consisting of
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by back translation; or
c) a functional equivalent of the nucleic acid sequence SEQ ID NO:1 with at least 52% identity with SEQ ID NO:1; or
d) a nucleic acid sequence which, on the basis of the degeneracy of the genetic code, can be deduced from the amino acid sequence of a functional equivalent of SEQ ID NO:2, which has at least 39% identity with SEQ ID NO:2, by back translation; where the polypeptide with the biological activity of an NADH-dependent cytochrome b5 reductase is overexpressed in the transgenic plant;
ii. applying a test substance to the transgenic plant of i) and to a nontransgenic plant of the same variety;
iii. determining the growth or the viability of the transgenic and of the nontransgenic plants after application of the test substance; and selection of test substances which bring about a modified growth of the nontransgenic plant in comparison with the growth of the transgenic plant.

15. A vehicle which has one or more of the nucleic acid molecules as claimed in any of claims 1 to 2, or one or more expression cassettes as claimed in claim 5, or one or more vectors as claimed in claim 6, or one or more organisms as claimed in claim 7 or one or more (poly)peptides as claimed in claim 3.

16. A method as claimed in any of claims 9 to 14, wherein the substances are identified in a high-throughput screening.

17. A herbicidally active compound identified by one or the methods as claimed in any of claims 9 to 13 and 16.

18. A growth-regulatory compound identified by the method as claimed in claims 14 and 16.

19. A process for the preparation of an agrochemical composition, which comprises
a) identifying a herbicidally active compound by one or the methods as claimed in any of claims 9 to 13 and 16 or a growth-regulatory compound as claimed in claims 14 and 16; and
b) formulating this compound together with suitable auxiliaries to give herbicidally or growth-regulatory crop protection products.

20. A method for controlling undesired vegetation and/or for regulating the growth of plants, which comprises allowing at least one compound as claimed in claim 17 or 18 or a compound obtainable by the method stated in claim 19 to act on plants, their environment and/or on seeds.

21. The use of a compound as claimed in claim 17 or 18 or of an agrochemical formulation obtainable by the method stated in claim 19 in a method as claimed in claim 20 for controlling undesired vegetation and/or for regulating the growth of plants.

22. A method for generating nucleic acid sequences which
i) encode a polypeptide with the biological activity of an NADH-dependent cytochrome b5 reductase which is not inhibited by substances as claimed in claim 17; and which are comprised by a functional equivalent of the nucleic acid sequence of the nucleic acid sequence SEQ ID NO:1 with at least 52% identity with SEQ ID NO:1;
which comprises the following process steps:
a) expression, in a heterologous system or in a cell-free system, of the proteins encoded by the nucleic acid according to i);
b) randomized or site-directed mutagenesis of the protein by modification of the nucleic acid;
c) measuring the interaction of the modified gene product with the herbicide;
d) identification of derivatives of the protein which show less interaction;
e) assaying the biological activity of the protein after application of the herbicide; and
f) selection of the nucleic acid sequences which show a modified biological activity toward the herbicide.

23. A method as claimed in claim 22, wherein the sequences selected in accordance with claim 22 f) are introduced into a nonhuman organism.

24. A method for generating transgenic plants which are resistant to substances as claimed in claim 17, which comprises overexpressing, in these plants, a nucleic acid sequence encoding a polypeptide with the biological activity of an NADH-dependent cytochrome b5 reductase which comprises
a) a nucleic acid sequence with the nucleic acid sequence shown in SEQ ID NO:1; or
b) a nucleic acid sequence which, on the basis of the degeneracy of the genetic code, can be deduced from the amino acid sequence shown in SEQ ID NO:2 by backtranslation; or
c) a nucleic acid sequence which, on the basis of the degeneracy of the genetic code, can be deduced from the amino acid sequence of a functional equivalent of SEQ ID NO:2 which has at least 39% identity with SEQ ID NO:2; or
d) a functional equivalent of the nucleic acid sequence SEQ ID NO:1 with at least 52% identity with SEQ ID NO:1.

25. A transgenic plant generated by a method as claimed in claim 24.

26. The use of compounds having an inhibitory or blocking effect on the activity of an NADH-dependent cytochrome 65 reductase to control undesired plant growth and/or regulate the growth of plants.
